(19) United States
(12) Patent Application Publication
(10) Pub. No.: US 2017/0100323 A1
(43) Pub. Date: Apr. 13, 2017

(54) Clear Compositions and Methods for the Delivery of Active Ingredients for Skin Care

(71) Applicant: Arbonne International, LLC, Irvine, CA (US)

(72) Inventor: Peter Matravers, Mission Viejo, CA (US)

(21) Appl. No.: 15/315,888

(22) PCT Filed: Jun. 4, 2015
(86) PCT No.: PCT/US15/34144

§ 371 Date: Dec. 2, 2016

Related U.S. Application Data
(60) Provisional application No. 62/007,837, filed on Jun. 4, 2014.

(51) Int. Cl. A61K 8/891; A61K 8/37; A61K 8/67; A61K 8/362; A61K 8/365; A61K 8/97; A61K 8/49; A61Q 19/00; A61Q 19/08; A61K 8/63; A61K 31/194; A61K 31/192; A61K 47/34; A61K 47/10; A61K 47/14; A61K 47/22; A61K 47/46; A61K 47/28; A61K 8/34

(52) U.S. Cl. CPC A61K 8/891; A61K 8/34; A61K 8/37; A61K 8/675; A61K 8/362; A61K 8/365; A61K 8/97; A61K 8/347; A61K 8/4973; A61Q 19/00; A61Q 19/08; A61K 8/63; A61K 31/194; A61K 31/192; A61K 47/34; A61K 47/10; A61K 47/14; A61K 47/22; A61K 47/46; A61K 47/28; A61K 2800/262

(57) ABSTRACT

Disclosed herein in some embodiments are compositions and methods for treating or cosmetically addressing skin conditions. In certain embodiments, the compositions utilize optically transparent carriers and are capable of dissolving hydrophilic and hydrophobic active ingredients. In an embodiment, the compositions comprise a volatile silicone, an organic alcohol, and a diester. In another embodiment, the compositions comprise a volatile silicone, specially-denatured ethanol, and diisopropyl sebacate. In another embodiment, the volatile silicone comprises a mixture of octamethyltrisiloxane and dimethicone. In other embodiments, the compositions are used to treat skin conditions. Methods of treating or improving skin firmness, skin hydration, skin wrinkles and fine lines, and skin clarity, among other methods, are disclosed.

### CROSS-REFERENCE TO RELATED APPLICATION

This application claims the benefit of U.S. Provisional Application Ser. No. 62/007,837 filed Jun. 4, 2014, which is incorporated herein by reference in its entirety.

### FIELD OF THE INVENTION

In some embodiments, the present invention relates to compositions and methods useful for skin care.

### BACKGROUND OF THE INVENTION

Formulations for topical delivery of active ingredients must simultaneously address multiple challenges. The impermeability of skin is well known, with the stratum corneum serving as a barrier against pathogens and toxic environmental chemicals. Optimal formulations may possess the ability to solubilize a wide range of hydrophobic and hydrophilic active ingredients, enhance the permeability of the active ingredients into the skin, and exhibit optimal physical properties of interest to the consumer such as, for example, an ideal evaporation rate and a non-greasy feel on the skin. There have been numerous attempts to make dermatological vehicles more aesthetically acceptable. However, formulations capable of solubilizing and delivering a mixture of hydrophilic and hydrophobic compounds using a carrier that is preferred by consumers and patients have been challenging to develop. In some cases, formulations, carriers, and vehicles that do not provide for solubility of hydrophobic and hydrophilic compounds in an optically clear composition may be less commercially desirable. In addition, formulations, carriers, and vehicles that not provide for good permeability across the skin, particularly through the stratum corneum layer may also be less commercially desirable in some cases.

There is a need for product compositions that can be used to deliver both hydrophobic and hydrophilic active ingredients wherein the properties of the carrier do not produce a greasy or oily feel and/or wherein the active ingredients have good retention even when the skin is moisturized, and which furthermore may allow for convenient and effective treatment or prevention of a wide variety of skin conditions and cosmetic needs.

### SUMMARY OF THE INVENTION

In an embodiment, a product composition includes a carrier composition that includes (a) a volatile silicone in a concentration of 40% by weight to 70% by weight of the product composition, (b) an organic alcohol in a concentration of 10% by weight to 30% by weight of the product composition, and (c) a compound according to Formula I

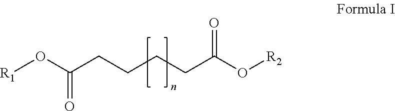

in a concentration of 1% by weight to 10% by weight of the product composition, wherein R₁ and R₂ are independently selected from the group consisting of isopropyl, propyl, and ethyl and wherein n is from 1 to 6, and wherein the carrier composition is an optically transparent liquid. In an embodiment, the product composition additionally includes active ingredients as defined herein.

In an embodiment, a method includes improving skin firmness in a human, improving skin hydration in a human, reducing mean fine line area in a human, reducing mean area of skin wrinkles in a human, and/or improving skin clarity in a human, the method comprising topically administering an optically transparent liquid product composition to the skin of the human.

### BRIEF DESCRIPTION OF THE DRAWINGS

The foregoing summary, as well as the following detailed description of the invention, will be better understood when read in conjunction with the appended drawings.

FIG. 1 illustrates the design of the carrier composition.

FIG. 2 illustrates the visible transmittance spectra of the carrier composition (“Carrier”), a product composition prepared according to Formula E as given in Table 5 (“Carrier with Active Ingredients”), and product compositions prepared according to Formula E as given in Table 5 but with the addition of 1% (“Carrier with Active Ingredients with 1% Added Water”), 1.5% (“Carrier with Active Ingredients with 1.5% Added Water”), and 2% water (“Carrier with Active Ingredients with 2% Added Water”).

FIG. 3 illustrates the UV-visible absorbance spectrum of a product composition prepared according to Formula E as given in Table 5 and diluted 1:100 vol/vol in specially-denatured alcohol (“Carrier with Active Ingredients, 1:100 Dilution in SD Alcohol”).

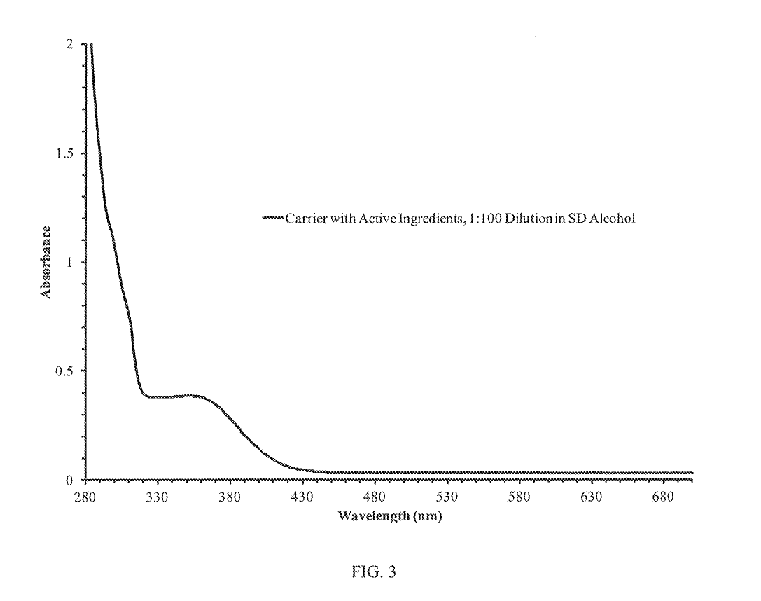

FIG. 4 illustrates photographs of a composition prepared according to Formula E as given in Table 5 (solution labeled 4), and compositions prepared according to Formula E as given in Table 5 but with the addition of 1% water (solution labeled 6), 1.5% water (solution labeled 7), and 2% water (solution labeled 8).

### DETAILED DESCRIPTION OF THE INVENTION

In the disclosure herein, compositions and methods for the delivery of active ingredients for skin care are described. As used herein, the term “product composition” means a composition suitable for use as a cosmetic or pharmaceutical composition for application to the skin or mucosal regions of a patient or subject. The term “by weight,” as used herein in relation to percentages or other quantities, may refer to the concentration of a component on a weight/weight basis (e.g. % w/w) or to the concentration of a component on a % weight/volume basis (e.g. % w/v). As used herein, the term “active ingredient” means a molecule, compound, or substance, including a mixture of molecules, compounds, or substances, which is suitable for delivery to the skin or mucosal regions of a patient or subject. As used herein, the term active ingredient also means a pharmaceutical, bioactive, natural, or cosmetic substance that may provide for the appearance of healthier skin. As used herein, an active ingredient may include cosmetic ingredients, ingredients that improve the appearance and/or texture of skin, bioactive ingredients, pharmaceutical active ingredients used to treat or prevent a disorder or condition, and ingredients used to improve consumer perception of a product. As used herein, active ingredients may include cosmetically elegant, non-greasy emollients that markedly enhance the delivery of other active ingredients across human skin, and may also improve the effectiveness of the ingredients in obtaining healthier skin, healthier-looking skin, and/or smooth-looking skin, and which also may provide excellent user compliance. As used herein, an active ingredient may include substances that provide for reduced wrinkles around the eyes, even tone skin without age spots, firmer, smoother skin texture, pore size reduction, moisturization, and hydration to skin, softer and more supple skin feel, a calming effect to reduce inflammation, prevention and treatment of acne, and whitening and/or brightening of skin appearance. Active ingredients are also referred to as active agents.

As used herein, the terms carrier and carrier composition both mean the base component of the composition used to deliver an active ingredient. In some embodiments, the carrier composition may also be considered to be a solvent for the active ingredients.

Many delivery systems that seek to deliver a mixture of hydrophilic and hydrophobic compounds make use of large concentrations of organic solvents, such as ethanol or isopropanol, for example as described in U.S. Pat. No. 4,826,828. Such delivery systems typically make use of >35% by weight of ethanol, as noted in U.S. Patent Application Publication No. 2013/0310355 A1, which is incorporated by reference herein in its entirety. Without being bound by any theory in the present application, the applicants in U.S. Patent Application Publication No. 2013/0310355 A1 have noted that such large concentrations of ethanol contribute to increased skin irritation and dryness, and can lead to non-optimal use by patients and a lack of consumer satisfaction.

U.S. Patent Application Publication No. 2013/0310355 A1 describes a delivery system for active agents including vitamin A that are not soluble in silicone oils. The delivery system comprises at least 50% by weight of ethyltrisiloxane (1,1,1,3,5,5,5-heptamethyl-3-ethyltrisiloxane) and less than 15% by weight of a volatile vehicle such as perfluorohexane, pentafluoropropane, perfluorohexane, perfluorodecalin, methoxynonafluorobutane, disiloxane, volatile high molecular weight hydrocarbons (such as isododecane), ethanol, or isopropyl alcohol at less than 15% by weight. Without being bound by any theory in the present application, the applicants in U.S. Patent Application Publication No. 2013/0310355 A1 have noted that the use of large amounts of ethyltrisiloxane leads to a product that causes undesirable drying of skin upon application.

In some embodiments, product compositions include compositions and methods for topical delivery of both water soluble active ingredients and water insoluble active ingredients to the skin. Some embodiments include compositions and methods for optimal delivery of ingredients with reduced drying, irritation, or inflammation. The ingredients may include, for example, retinoids, antibacterials, alpha-hydroxy acids, beta-hydroxy acids, antibiotics, aqueous plant extracts, moisturizers, skin calming agents, skin rejuvenating agents, vitamins A, B, C, D, and E, corticosteriods, anti-inflammatory compounds, skin whitening ingredients, sunscreen agents for protection from ultraviolet radiation, skin smoothing agents and other active ingredients to the skin. Some embodiments include compositions that comprise an optically clear carrier for delivery of hydrophobic and hydrophilic ingredients. Some embodiments include methods comprising the steps of pouring of an optically transparent carrier or a product composition described herein over a pad and applying the pad to the skin to achieve delivery of the desired ingredients within the composition to address at least one skin condition (e.g., by enhancing appearance or by treating the condition). Some embodiments include methods of preventing and/or treating skin conditions or disorders. Other embodiments include methods of preventing and/or treating acne. Further embodiments include methods of reducing skin blemishes, reducing skin discoloration, reducing mean fine line area, reducing mean area of skin wrinkles, improving skin firmness, improving skin hydration, improving skin clarity and/or by giving the appearance of one of the foregoing results. In one embodiment, the present disclosure includes compositions and methods for topical delivery of both water soluble active ingredients and water insoluble ingredients (e.g., active ingredients) to the skin. In one embodiment, the present disclosure includes compositions and methods for optimal delivery to the skin of retinoid compounds, alpha-hydroxy acids, beta-hydroxy acids, natural ingredients, antiseptic agents, antibacterial agents, antibiotic agents, anti-inflammatory agents, antiviral agents, antifungal agents, sunscreen agents, skin whitening agents, plant extracts, vitamins, corticosteroids, local anesthetic agents, other bioactive ingredients, pigments, and/or ingredients that improve or alter the consumer perception of a product. In another embodiment, the present disclosure includes compositions and methods for optimal delivery of aqueous plant extracts, moisturizers, skin calming agents, skin rejuvenating agents, and other ingredients to the skin with reduced drying, irritation, or inflammation. In one embodiment, the present disclosure includes compositions and methods that use a clear carrier for delivery of hydrophobic and hydrophilic ingredients. In another embodiment, the present disclosure provides methods that include pouring of the clear carrier over a pad, and application of the solution to the skin using the pad.

One embodiment includes a preparation of anhydrous product compositions for delivery of active ingredients to the skin that is substantially free of clays, starches, polysaccharides or celluloses as a replacement for the oils in anhydrous systems. In an embodiment, a dry-feeling product composition is achieved that does not suffer from poor penetration of active ingredients into the skin. In some embodiments, clays, starches, polysaccharides or celluloses are used in small amounts. In some embodiments, larger amounts can cause properties in the final products such as stickiness, a pasty character, and agglomeration of particulates. In an embodiment, a product composition includes a starch at a level of 3% by weight to 30% by weight. In an embodiment, a product composition includes a starch at a level of 7.5% by weight. In an embodiment, a product composition includes a starch at a level of 10% by weight. In an embodiment, a product composition includes an anhydrous, hydrophobic “dry flow” starch. Suitable starches are also described in U.S. Pat. No. 4,894,222, the disclosure of which is incorporated herein by reference.

Another embodiment includes a preparation of anhydrous product compositions for delivery of active ingredients to the skin that re substantially free of mineral oils, petrolatum, and animal and vegetable fats and oils as dermatological vehicles or cosmetic carriers. Another embodiment includes a preparation of anhydrous product compositions for delivery of active ingredients to the skin that is substantially free of an ointment, cream, or lotion. In an embodiment, a dry-feeling product composition is achieved that does not suffer from a greasy feel as experienced by a consumer. In some embodiments, larger amounts of mineral oils, petrolatum, and animal and vegetable fats and oils as dermatological vehicles or cosmetic carriers can cause the final product to exhibit a greasy feel. In an embodiment, a product composition includes a mineral oil at a level of 0% by weight to 90% by weight. In an embodiment, a product composition includes a mineral oil at a level of 1% by weight to 10% by weight. In an embodiment, a product composition includes a mineral oil at a level of 7% by weight. Suitable mineral oils are also described in U.S. Pat. No. 4,894,222, the disclosure of which is incorporated herein by reference.

As set forth in detail elsewhere herein, it has been found that embodiments of the product compositions described herein as administered to a subject according to methods embodied herein provide surprising efficacy for treatment of skin conditions and other disorders and also demonstrate an unexpected ability to solubilize hydrophobic and hydrophilic active ingredients. In one embodiment, there is a product composition that is configured for application to skin. The product composition may be a liquid and/or colorless.

### Carriers

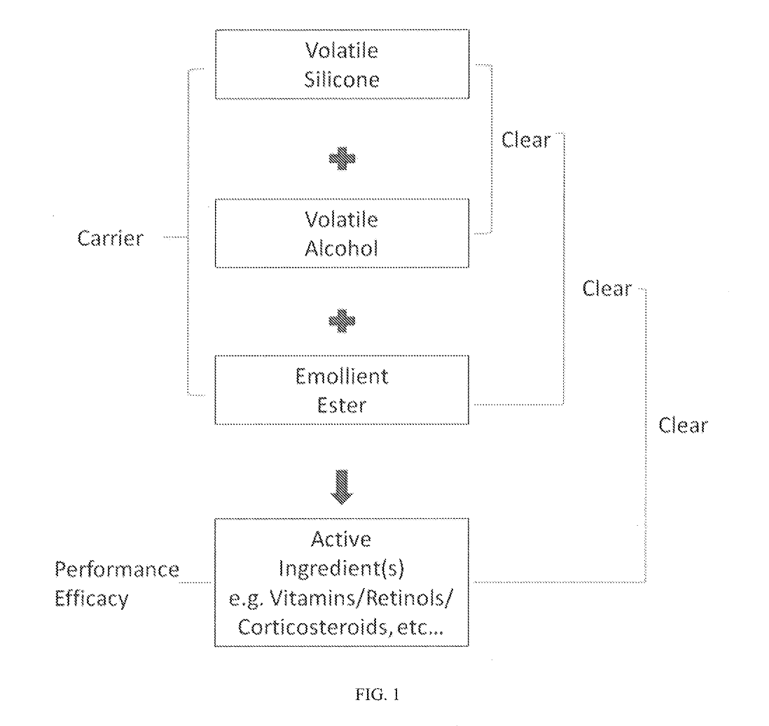

The design of a carrier composition, also referred to as a carrier, is depicted in FIG. 1. In some embodiments, the product compositions disclosed here include a carrier composition. The carrier composition may serve as the base component of the compositions described herein. The carrier composition may provide the vehicle by which the active ingredients are solublized and delivered to the skin. The carrier composition may also be referred to as a “base,” “vehicle,” or “transport vehicle” for a skin care composition. Within the embodiments of product compositions described herein, in one embodiment the carrier composition provides the means for delivery of the active ingredients. In an embodiment, the product compositions disclosed here include a carrier composition, wherein the carrier composition comprises at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight of the product composition. In an embodiment, the product compositions disclosed here include a carrier composition, wherein the carrier composition comprises at least 85% to 90% by weight of the composition. In another embodiment, a composition includes a carrier composition, wherein the carrier composition comprises at least 90% to 95% by weight of the product composition. In another embodiment, a product composition includes a carrier composition, wherein the carrier composition comprises at least 95% to 99% by weight of the composition. In another embodiment, a product composition includes a carrier composition, wherein the carrier composition comprises at least 90% to 93% by weight of the composition. In another embodiment, a product composition includes a carrier composition, wherein the carrier composition comprises at least 93% to 97% by weight of the composition. In another embodiment, a product composition includes a carrier composition, wherein the carrier composition comprises at least 97% to 99% by weight of the composition.

Exemplary carriers may comprise at least one organic alcohol. Suitable organic alcohols include, for example, ethanol, specially-denatured (SD) alcohol, isopropanol, and mixtures thereof. A preferred embodiment includes SD alcohol. A preferred embodiment includes SD alcohol, where the SD alcohol includes ethanol and a denaturing compound added to render the ethanol unsuitable for drinking A preferred embodiment includes SD alcohol, where the SD alcohol includes ethanol and ethyl acetate. In an embodiment, the organic alcohol may include a mixture of about 50% by weight of ethanol and about 50% by weight of isopropanol. In an embodiment, the organic alcohol may include a mixture of about 40% by weight of ethanol and about 60% by weight of isopropanol. In an embodiment, the organic alcohol may include a mixture of about 60% by weight of ethanol and about 40% by weight of isopropanol. In an embodiment, the organic alcohol may include a mixture of about 30% by weight of ethanol and about 70% by weight of isopropanol. In an embodiment, the organic alcohol may include a mixture of about 70% by weight of ethanol and about 30% by weight of isopropanol. In an embodiment, the organic alcohol may include a mixture of about 25% by weight of ethanol and about 75% by weight of isopropanol. In an embodiment, the organic alcohol may include a mixture of about 75% by weight of ethanol and about 25% by weight of isopropanol. In an embodiment, the organic alcohol may include a mixture of about 20% by weight of ethanol and about 80% by weight of isopropanol. In an embodiment, the organic alcohol may include a mixture of about 80% by weight of ethanol and about 20% by weight of isopropanol. In an embodiment, the organic alcohol may include a mixture of about 10% by weight of ethanol and about 90% by weight of isopropanol. In an embodiment, the organic alcohol may include a mixture of about 90% by weight of ethanol and about 10% by weight of isopropanol. In an embodiment, the organic alcohol may include a mixture of 10% by weight of ethanol to 90% by weight of isopropanol in combination with ethanol. In an embodiment, the organic alcohol may include a mixture of 20% by weight of ethanol to 80% by weight of isopropanol in combination with ethanol. In an embodiment, the organic alcohol may include a mixture of 30% by weight of ethanol to 70% by weight of isopropanol in combination with ethanol. In an embodiment, the organic alcohol may include a mixture of 40% by weight of ethanol to 60% by weight of isopropanol in combination with ethanol. In an embodiment, the organic alcohol may include an organic alcohol and a denaturant (e.g. specially-denatured ethanol or SD-40 alcohol).

A carrier may also include an emollient ester. Emollient esters, when applied to skin, may assist in the prevention of loss of skin hydration, serve as thickening agents, provide lubrication, and improve tactile perception. The preferred emollient esters are compositions including the compound of Formula I.

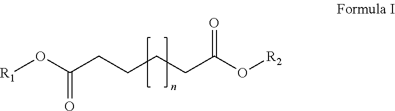

wherein R₁ and R₂ are independently selected from the group consisting of isopropyl, propyl, and ethyl, and wherein n is an integer from 1 to 6. Examples of suitable emollient esters include diisopropyl sebacate, diisopropyl nonanedioate, diisopropyl octanedioate, diisopropyl heptanedioate, diisopropyl adipate, and combinations thereof.

A preferred emollient ester is diisopropyl sebacate (Formula II), also known as diisopropyl decanedioate and by its trade name DUB DIS, which has been found to demonstrate excellent compatibility in hydro-alcoholic and anhydrous cosmetic formulations.

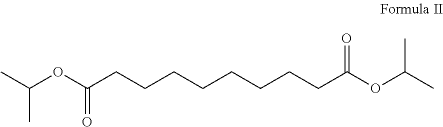 [SMILES: CC(C)OC(=O)CCCCCCCCC(=O)OC(C)C]

Diisopropyl sebacate may be used in facial lotions as a non-oily emollient lubricant with quick drying effects and a fast spreading action. Surprisingly, in an embodiment, diisopropyl sebacate was employed to provide excellent solubilizing and penetration-enhancing properties as well as emollient properties in a clear, pourable formulation with fast evaporation suitable for use with a pad application.

Another preferred emollient ester is diisopropyl adipate (Formula III), which is also known as diisopropyl hexanedioate.

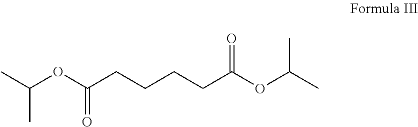 [SMILES: CC(C)OC(=O)CCCCC(=O)OC(C)C]

In an embodiment, an emollient ester is selected from the group consisting of decyl benzoate, undecyl benzoate, dodecyl benzoate, tridecyl benzoate, tetradecyl benzoate, pentadecyl benzoate, hexadecyl benzoate, cetyl esters, caprylic/capric diglyceryl succinate, diethylhexyl malate, ethyl hexyl palmitate, neopentyl glycol dicaprate, ethylene glycol dicaprate, castor oil benzoate (e.g. FINSOLV BCO-115), ethylhexyl hydroxystearate benzoate (e.g. FINSOLV BOHS-111), C12-15 alkyl benzoate (e.g. FINSOLV TN or FINSOLV TN-O), dipropylene glycol dibenzoate (e.g. FINSOLVE PG-22), methyl gluceth-20 benzoate (e.g. FINSOLVE EMG-20), a mixture of C12-15 alkyl benzoate and dipropylene glycol dibenzoate and PPG-15 stearyl ether benzoate (e.g. FINSOLV TPP), dimethicone PEG/PPG-20/23 benzoate (e.g. FINSOLV SLB-101), dimethicone PEG-8 benzoate (e.g. FINSOLV SLB-201), ethylhexyl benzoate (e.g. FINSOLV EB), combinations thereof, combinations thereof with compounds of Formula I, combinations thereof with diisopropyl sebacate, and combinations thereof with diisopropyl adipate. Other alkyl benzoates and esters may also be useful in some embodiments.

In one embodiment, suitable carriers include at least one volatile silicone. Volatile silicones may also be referred to as silicone oils and organic siloxanes by those of ordinary skill in the art. Suitable volatile silicones are described in U.S. Patent Publication Nos. 2004/0197284 A1, 2009/0074689 A1, 2012/0129956 A1, 2002/0022040 A1, 2003/0049212 A1, 2008/0044494 A1, and U.S. Pat. No. 5,300,286, the disclosures of which are incorporated by reference herein.

In an embodiment, a suitable volatile silicone may include the organic siloxane of Formula IV:

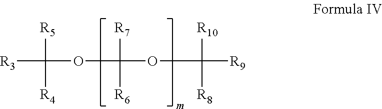

wherein R₃, R₄, R₅, R₆, R₇, R₈, R₉, and R₁₀ are independently selected from the group consisting of alkyl, aryl, and alkoxyl groups, and m is an integer from 1 to 8. In a preferred embodiment, one or more of R₃, R₄, R₅, R₆, R₇, R₈, R₉, and R₁₀ are methyl groups and m is an integer from 1 to 6. In a most preferred embodiment, R₃, R₄, R₅, R₆, R₇, R₈, R₉, and R₁₀ are methyl groups and m is an integer from 1 to 4.

A preferred volatile silicone includes the organic siloxane of Formula V:

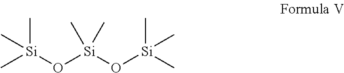

The volatile siloxane of Formula V is also known as octamethyltrisiloxane or synonymously as trisiloxane. Trisiloxane is a component of commercial silicone oil blends, such as the XIAMETER PMX-1184 blend or 2-1184 silicone fluid blend (both available from Dow Corning, Midland, Mich., USA). In an embodiment, the volatile silicone includes trisiloxane and/or other related substances as described in U.S. Patent Publication Nos. 2004/0197284 A1 and 2009/0074689 A1, the disclosures of which are incorporated by reference herein.

A preferred volatile silicone includes the organic siloxane of Formula VI:

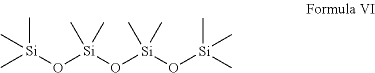

The volatile siloxane of Formula VI is also known as decamethyltetrasiloxane or synonymously as tetrasiloxane. Tetrasiloxane is a component of commercial silicone oil blends, such as the XIAMETER PMX-1184 blend (Dow Corning, Midland, Mich., USA). In such blends, the quantity of tetrasiloxane may be referred to as dimethicone. In another embodiment, the volatile silicone includes tetrasiloxane and/or other related substances as described in U.S. Patent Publication Nos. 2004/0197284 A1 and 2009/0074689 A1, the disclosures of which are incorporated by reference herein.

A preferred volatile silicone includes the organic siloxane of Formula VII:

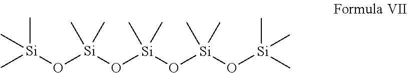

The volatile siloxane of Formula VII is also known as dodecamethylpentasiloxane or synonymously as pentasiloxane. Pentasiloxane is a component of commercial silicone oil blends, such as the XIAMETER PMX-1184 blend (Dow Corning, Midland, Mich., USA). In such blends, the quantity of pentasiloxane may be referred to as dimethicone. In another embodiment, the volatile silicone includes pentasiloxane and/or other related substances as described in U.S. Patent Publication Nos. 2004/0197284 A1 and 2009/0074689 A1, the disclosures of which are incorporated by reference herein.

In a preferred embodiment, the volatile silicone includes dimethicone. Dimethicone is a mixture of methylated polysiloxanes and is described, for example, in U.S. Pat. No. 2,441,098, the disclosure of which is incorporated by reference herein in its entirety. The use of the term dimethicone also commonly includes tetrasiloxane and pentasiloxane. Some applications, properties, and uses of dimethicone in skin care products are described in Disapio, A.; Fridd, P. Int. J. Cosmet. Sci. 1988, 10, 75-89. In an embodiment, the volatile silicone includes dimethicone. In a preferred embodiment, the volatile silicone includes a mixture of dimethicone and trisiloxane. In another preferred embodiment, the volatile silicone includes a mixture that includes 30 to 70% by weight of dimethicone and 30 to 70% by weight of trisiloxane.

In another embodiment, the volatile silicone includes other siloxanes and related substances, including solubilizers, as described in U.S. Patent Application Publication Nos. 2014/0010769, 2013/0310355 A1, 2004/0197284 A1 and 2009/0074689 A1, the disclosures of which are incorporated by reference herein.

In another embodiment, the volatile silicone includes 1,1,1,3,5,5,5-heptamethyl-3-ethyltrisiloxane, also known as ethyltrisiloxane, which corresponds to Formula IV when m=1, R₇ is an ethyl group, and R₃, R₄, R₅, R₆, R₈, R₉, and R₁₀ are methyl groups. Commercial ethyltrisiloxane may be used, such as the SILSOFT ETS product (Momentive Performance Materials, Columbus, Ohio, USA). U.S. Patent Application Publication No. 2013/0310355 A1, the disclosure of which is incorporated herein by reference, describes the use of ethyltrisiloxane in a delivery system for active agents, including retinoids, which may not be soluble in silicone oils.

In another embodiment, the volatile silicone includes disiloxane, which is also known as hexamethyldisiloxane. Disiloxane provides for rapid evaporation and other useful properties, and alone or in combination with other materials can provide a composition with optimal characteristics. In another embodiment, the volatile silicone includes disiloxane and/or other related substances as described in U.S. Patent Publication Nos. 2004/0197284 A1 and 2009/0074689 A1, the disclosures of which are incorporated by reference herein. In another embodiment, the volatile silicone includes disiloxane, trisiloxane, tetrasiloxane, pentasiloxane, and/or dimethicone.

In another embodiment, the volatile silicone includes a cyclosiloxane, such as hexamethylcyclotrisiloxane (also known as cyclotrisiloxane or D3), octamethylcyclotetrasiloxane (also known as cyclotetrasiloxane or D4), decamethylcyclopentasiloxane (also known as cyclopentasiloxane or D5), or dodecamethylcyclohexasiloxane (also known as cyclohexasiloxane or D6), tetradecamethylcycloheptasiloxane (D7), or mixtures thereof. In another embodiment, the volatile silicone includes compounds of the general formula (CH₃)₂ₚOₚSiₚ where p in an integer from 3 to 7. In another embodiment, the volatile silicone includes cyclomethicone. Commercial cyclosiloxane blends may be used in some embodiments, such as the cyclohexasiloxane blend with trade name XIAMETER PMX-0345 (Dow Corning, Midland, Mich., USA). In another embodiment, the volatile silicone includes a cyclosiloxane, trisiloxane, tetrasiloxane, pentasiloxane, and/or dimethicone.

In an embodiment, the volatile silicone includes blends of organic siloxanes, including blended dimethicones such as XIAMETER PMX-200 silicone fluid and XIAMETER PMX-0225 silicone fluid (Dow Corning, Midland, Mich., USA). In another embodiment, the volatile silicone includes an amodimethicone such as XIAMETER OFX-8220 fluid (Dow Corning, Midland, Mich., USA). In another embodiment, the volatile silicone includes a blend of cyclopentasiloxane and trimethylsiloxysilicate diluted in cyclopentasiloxane, such as the commercially available XIAMETER RSN-0749 resin (Dow Corning, Midland, Mich., USA). In another embodiment, the volatile silicone includes a blend of cyclopentasiloxane and trimethylsiloxysilicate diluted in cyclopentasiloxane, in addition to trisiloxane, tetrasiloxane, pentasiloxane, and/or dimethicone.

In an embodiment, the volatile silicone includes phenyl trimethicone, bis-phenyl trimethicone, silicone resin SR1000 (Momentive Performance Materials, Columbus, Ohio, USA), and/or silicone resin SF1318 (Momentive Performance Materials, Columbus, Ohio, USA). In another embodiment, the volatile silicone includes phenyl trimethicone, bis-phenyl trimethicone, or silicone resin, in addition to trisiloxane, tetrasiloxane, pentasiloxane, and/or dimethicone.

Blends of any of the volatile silicones described above may be employed in some embodiments to achieve superior results with respect to drying time, distribution, tactile feel, and other key properties of a skin care composition. In an embodiment, a volatile silicone includes 30% by weight to 50% by weight of decamethyltetrasiloxane, 30% by weight to 50% by weight of octamethyltrisiloxane, 10% by weight to 35% by weight of dodecamethylpentasiloxane, and 10% by weight to 30% of polydimethylsiloxane. In another embodiment, a volatile silicone includes 30% by weight to 45% by weight of decamethyltetrasiloxane, 25% by weight to 40% by weight of octamethyltrisiloxane, 12% by weight to 20% by weight of dodecamethylpentasiloxane, and 12% by weight to 20% of polydimethylsiloxane. In a preferred embodiment, a volatile silicone includes about 32 to 38% by weight of decamethyltetrasiloxane, about 29 to 35% by weight of octamethyltrisiloxane, 12% by weight to 20% by weight of dodecamethylpentasiloxane, 12% by weight to 20% of polydimethylsiloxane, and less than 2% of octamethylcyclotetrasiloxane. In a preferred embodiment, a volatile silicone includes about 35% by weight of decamethyltetrasiloxane, about 32% by weight of octamethyltrisiloxane, 15% by weight to 18% by weight of dodecamethylpentasiloxane, 15% by weight to 18% of polydimethylsiloxane, and less than 1% of octamethylcyclotetrasiloxane. In an embodiment, any of the foregoing volatile silicone compositions may additionally include less than 0.5%, 1%, 2% or 3% of octamethylcyclotetrasiloxane.

In an embodiment, a carrier composition includes: (1) an organic alcohol; (2) a volatile silicone; and (3) a compound of Formula I. In an embodiment, a carrier composition includes: (1) 10 to 30% of an organic alcohol, (2) 30 to 80% of a volatile silicone; and (3) 1 to 10% of a compound of Formula I. In an embodiment, a carrier composition includes: (1) 10 to 30% of an organic alcohol, (2) 40 to 70% of a volatile silicone; and (3) 1 to 10% of a compound of Formula I. In an embodiment, a carrier composition includes: (1) 20 to 30% of an organic alcohol, (2) 40 to 70% of a volatile silicone; and (3) 2 to 8% of a compound of Formula I. In an embodiment, a carrier composition includes: (1) 10 to 30% of SD alcohol; (2) 40 to 70% of a volatile silicone, where the volatile silicone is a blend that includes 32 to 38% by weight of decamethyltetrasiloxane, 29 to 35% by weight of octamethyltrisiloxane, 12% by weight to 20% by weight of dodecamethylpentasiloxane, 12% by weight to 20% of polydimethylsiloxane, and less than 2% of octamethylcyclotetrasiloxane; and (3) 1 to 10% of a compound of Formula II. In an embodiment, a carrier composition includes: (1) 10 to 30% of SD alcohol; (2) 40 to 70% of a volatile silicone, where the volatile silicone is a blend that includes about 35% by weight of decamethyltetrasiloxane, about 32% by weight of octamethyltrisiloxane, 15% by weight to 18% by weight of dodecamethylpentasiloxane, 15% by weight to 18% of polydimethylsiloxane, and less than 1% of octamethylcyclotetrasiloxane; and (3) 1 to 10% of diisopropyl sebacate (Formula II). In an embodiment, a carrier composition includes: (1) 20 to 30% SD alcohol; (2) 40 to 70% of a volatile silicone, where the volatile silicone is a blend that includes about 35% by weight of decamethyltetrasiloxane, about 32% by weight of octamethyltrisiloxane, 15% by weight to 18% by weight of dodecamethylpentasiloxane, 15% by weight to 18% of polydimethylsiloxane, and less than 1% of octamethylcyclotetrasiloxane; and (3) 2 to 8% of diisopropyl sebacate (Formula II). In an embodiment, a carrier composition includes (1) SD alcohol, (2) a volatile silicone (where the volatile silicone is a blend that includes about 35% by weight of decamethyltetrasiloxane, about 32% by weight of octamethyltrisiloxane, 15% by weight to 18% by weight of dodecamethylpentasiloxane, 15% by weight to 18% of polydimethylsiloxane, and less than 1% of octamethylcyclotetrasiloxane) and (3) diisopropyl sebacate (Formula II), where the weight:weight:weight ratio of SD alcohol:volatile silicone:diisopropyl sebacate is selected from the group consisting of 19:70:1, 20:69:1, 21:68:1, 22:67:1, 23:66:1, 24:65:1, 25:64:1, 26:63:1, 27:62:1, 28:61:1, 29:60:1, 30:59:1, 19:70:2, 20:69:2, 21:68:2, 22:67:2, 23:66:2, 24:65:2, 25:64:2, 26:63:2, 27:62:2, 28:61:2, 29:60:2, 30:59:2, 19:70:3, 20:69:3, 21:68:3, 22:67:3, 23:66:3, 24:65:3, 25:64:3, 26:63:3, 27:62:3, 28:61:3, 29:60:3, 30:59:3, 19:70:4, 20:69:4, 21:68:4, 22:67:4, 23:66:4, 24:65:4, 25:64:4, 26:63:4, 27:62:4, 28:61:4, 29:60:4, 30:59:4, 19:70:5, 20:69:5, 21:68:5, 22:67:5, 23:66:5, 24:65:5, 25:64:5, 26:63:5, 27:62:5, 28:61:5, 29:60:5, 30:59:5, 19:70:6, 20:69:6, 21:68:6, 22:67:6, 23:66:6, 24:65:6, 25:64:6, 26:63:6, 27:62:6, 28:61:6, 29:60:6, 30:59:6, 19:70:7, 20:69:7, 21:68:7, 22:67:7, 23:66:7, 24:65:7, 25:64:7, 26:63:7, 27:62:7, 28:61:7, 29:60:7, 30:59:7, 19:70:8, 20:69:8, 21:68:8, 22:67:8, 23:66:8, 24:65:8, 25:64:8, 26:63:8, 27:62:8, 28:61:8, 29:60:8, 30:59:8, 19:70:9, 20:69:9, 21:68:9, 22:67:9, 23:66:9, 24:65:9, 25:64:9, 26:63:9, 27:62:9, 28:61:9, 29:60:9, 30:59:9, 19:70:10, 20:69:10, 21:68:10, 22:67:10, 23:66:10, 24:65:10, 25:64:10, 26:63:10, 27:62:10, 28:61:10, 29:60:10, and 30:59:10.

In an embodiment, the carrier composition is substantially anhydrous. The carrier compositions may also be miscible with small amounts of water. In some embodiments, and without wishing to be bound by any particular theory, the small amounts of water allow for the use of active ingredients that contain water because of hygroscopicity, water of hydration, or as a component of a blend. This may provide an advantage over other formulations. In an embodiment, a composition includes a carrier composition with 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0% by weight of water. In an embodiment, the composition includes a carrier composition with less than 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0% by weight of water. In an embodiment, the compositions include 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0% by weight of water. In an embodiment, the compositions include a colorless liquid composition comprising less than 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0% by weight of water. Preferably the selected carrier is included a composition that is a colorless and/or liquid composition.

### Evaporation Rate of Compositions

Embodiments of the carriers and/or the product compositions described herein may have an evaporation rate designed to provide an optimal consumer experience. Suitable evaporation rates of carriers and product compositions are fluids that may evaporate rapidly on contact with skin. In an embodiment, evaporation rates of carriers and product compositions are fluids that may evaporate on contact with the skin in less than 1 minute, 5 minutes, 15 minutes, 30 minutes, and/or one hour in an ambient environment at room temperature (25° C.) and at atmospheric pressure (760 torr). In an embodiment, evaporation rates of carriers and product compositions range from 10 to 500 mg/cm²/min at room temperature and atmospheric pressure. In another embodiment, evaporation rates of carriers and product compositions range from 20 to 200 mg/cm²/min at room temperature and atmospheric pressure. In an embodiment, evaporation rates of carriers and product compositions range from 100 to 200 mg/cm²/min at room temperature and atmospheric pressure. In another embodiment, evaporation rates of carriers and product compositions range from 200 to 500 mg/cm²/min at room temperature and atmospheric pressure.

Clear and/or Colorless Compositions

Embodiments of the carriers and/or the product compositions described herein may be an optically clear liquid. An optically clear liquid may provide both aesthetic and functional advantages over turbid or opaque compositions. In an embodiment, a composition includes a carrier composition that is a substantially colorless liquid composition. In an embodiment, a composition includes a carrier and/or a product composition that is optically clear to the naked eye. In an embodiment, a composition includes a carrier and/or a product composition that appears as an optically clear, lightly-colored liquid to the naked eye. In an embodiment, a composition includes a carrier and/or a product composition that appears to the naked eye as an optically clear liquid with a pale yellow color.

Optical clarity may be measured by well established methods that measure the transmittance of a solution and the turbidity of a solution. In an embodiment, a composition includes a carrier composition that is substantially free of absorbance between 300 and 700 nm. In an embodiment, a composition includes a carrier composition that has a transmittance of greater than 90% between 400 and 700 nm. In an embodiment, a composition includes a carrier composition that has a transmittance of greater than 85% between 400 and 700 nm. In an embodiment, a composition includes a carrier composition that has a transmittance of greater than 80% between 400 and 700 nm. In an embodiment, a composition includes a carrier composition that has a transmittance of greater than 75% between 400 and 700 nm.

In an embodiment, a composition includes a carrier composition that has a transmittance of greater than 50% at 450 nm. In an embodiment, a composition includes a product composition that has a transmittance of greater than 75% at 500 nm. In an embodiment, a composition includes a product composition that has a transmittance of greater than 85% at 550 nm. In an embodiment, a composition includes a product composition that has a transmittance of greater than 90% at 600 nm. In an embodiment, a composition includes a product composition that has a transmittance of greater than 90% at 650 nm. In an embodiment, a composition includes a product composition that has a transmittance of greater than 90% at 700 nm.

In an embodiment, a composition includes a product composition that has a transmittance of between 75% and 90% at 500 nm. In an embodiment, a composition includes a product composition that has a transmittance of between 80% and 95% at 550 nm. In an embodiment, a composition includes a product composition that has a transmittance of between 80% and 95% at 600 nm. In an embodiment, a composition includes a product composition that has a transmittance of between 80% and 95% at 650 nm. In an embodiment, a composition includes a product composition that has a transmittance of between 80% and 95% at 700 nm. In an embodiment, a composition includes a product composition that has a transmittance of between 90% and 95% at 550 nm. In an embodiment, a composition includes a product composition that has a transmittance of between 90% and 95% at 600 nm. In an embodiment, a composition includes a product composition that has a transmittance of between 90% and 95% at 650 nm. In an embodiment, a composition includes a product composition that has a transmittance of between 90% and 95% at 700 nm.

In one embodiment, the carrier is optically clear. In an embodiment, a carrier composition is colorless, optically transparent, or optically clear to the human eye. In an embodiment, a product composition is optically clear to the human eye and possesses a pale yellow color. In another embodiment, a product composition is optically clear to the human eye and possesses a light yellow color. In another embodiment, a product composition is optically clear to the human eye and possesses a color selected from the group consisting of a light red color, a light orange color, a light yellow color, a light pink color, a light green color, a light blue color, and a light purple color. In another embodiment, a product composition is optically clear to the human eye and is slightly opalescent. In another embodiment, a product composition is optically clear to the human eye and is slightly turbid.

### Active Ingredients

The aforementioned carriers may be combined with an active ingredient or a combination of active ingredients. Active ingredients of use with the compositions and methods described here may include those active ingredients that are known to those of ordinary skill in the art, such as those described in: Draelos, Z. D., Active agents in skin care products, Plast. Reconstru. Surg., 2010, 125, 719-724. Non-limiting examples of active ingredients suitable for use with the compositions and methods are described herein. A composition may include one or more hydrophobic (i.e. oil-soluble) or hydrophilic (i.e. water-soluble) active ingredients that are known to those of ordinary skill in the art. Active ingredients also include salts thereof, including pharmaceutically-acceptable salts, cocrystals thereof, complexes thereof, or derivatives thereof, including prodrugs such as esters and phosphate esters, as described herein or known to those of ordinary skill in the art.

The active ingredients in the compositions and methods described herein may include retinoid compounds. The preferred retinoid compounds include hydrophobic compounds. Suitable retinoid compounds include vitamin A, vitamin A derivatives, retinal, retinoic acid, retinal ester, retinol, tretinoin or esters thereof, isotretinoin or esters thereof, trans-retinoic acid or derivatives thereof, beta-carotene, beta-carotene derivatives, adapalene, tazarotene, or combinations thereof, and salts thereof, including pharmaceutically-acceptable salts, cocrystals thereof, complexes thereof, or other derivatives thereof, including prodrugs. In a preferred embodiment, a retinoid compound includes hydroxypinacolone retinoate (HPR). Other active ingredients described elsewhere in this specification may also be retinoid compounds and are hereby included as such in the scope of this disclosure. The active ingredients in the compositions and methods described herein may include alternatives to retinoid compounds (e.g. retinoid-like compounds) that function in a similar manner, including meroterpene phenols. The preferred retinoid-like compounds include bakuchiol (4-[(1E,3S)-3-ethenyl-3,7-dimethyl-1,6-octadien-1-yl]phenol), which is described in: Chaudhuri, R. K., Bojanowski, K. Bakuchiol: a retinol-like functional compound revealed by gene expression profiling and clinically proven to have anti-aging effects. Int. J. Cosmet. Sci. 2014, 36, 221-230.

The active ingredients in the compositions and methods described herein may include skin penetration enhancers. Skin penetration enhancers are commonly used with retinoid compounds as well as other active ingredients described herein. The preferred skin penetration enhancer is dimethyl isosorbide. Suitable skin penetration enhancers for use with the compositions and methods described here may include skin penetration enhancers that are known to those of ordinary skill in the art, such as those described in: Williams, A. C., Barry, B. W. Penetration enhancers, Adv. Drug. Deliv. Rev. 2004, 56, 603-618. Suitable skin penetration enhancers include sulfoxides (such as dimethylsulfoxide), azones (such as laurocapram and 1-dodecylazacycloheptan-2-one), pyrrolidones (such as 2-pyrrolidone), alcohols (such as ethanol or decanol), glycols (such as propylene glycol), surfactants (including alkyl carboxylates and their corresponding acids such as oleic acid, fluoroalkylcarboxylates and their corresponding acids, alkyl sulfates, alkyl ether sulfates, docusates such as dioctyl sodium sulfosuccinate, alkyl benzene sulfonates, alkyl ether phosphates, and alkyl aryl ether phosphates), and terpenes (such as limonene, p-cymene, geraniol, farnesol, eugenol, menthol, terpineol, carveol, carvone, fenchone, and verbenone).

Active ingredients in the compositions and methods described herein may also include alpha-hydroxy acids, which are also referred to as alpha-hydroxycarboxylic acids. Alpha-hydroxy acids, such as glycolic acid, may provide skin moisturization and exfoliation properties. Alpha-hydroxy acids can penetrate the upper layer of the skin quickly using a suitable carrier and weaken the anchors that hold dead skin cells together, allowing for easy removal of layers of dead skin cells, which in turn allows for rejuvenation and reveals the smoother and more evenly pigmented underlying skin. Alpha-hydroxy acids may thus provide for improved skin tone, color, and texture. In addition, alpha-hydroxy acids help to bind water to the skin to increase skin hydration and provide for a healthy complexion. In an embodiment, a product composition or carrier includes an alpha-hydroxy acid selected from the group consisting of glycolic acid, lactic acid, malic acid, citric acid, tartaric acid, pyruvic acid, and combinations thereof, and salts thereof, including pharmaceutically-acceptable salts, cocrystals thereof, complexes thereof, or derivatives thereof, including prodrugs. In a preferred embodiment, a product composition or carrier includes glycolic acid. Suitable alpha-hydroxy acids are also described in U.S. Pat. No. 6,521,271, the disclosure of which is incorporated herein by reference. Other active ingredients described elsewhere in this specification may also be alpha-hydroxy acids and are hereby included as such in the scope of this disclosure.

Active ingredients in the compositions and methods described herein may also include beta-hydroxy acids, which are also referred to as beta-hydroxycarboxylic acids. Beta-hydroxy acids, such as salicylic acid, are known for its antimicrobial properties among other useful properties that enable their use as a remedy for problem skin conditions. NAB willow bark extract (Lonza Ltd., Basel, Switzerland), also known as aqueous Salix Nigra (Willow) Bark Extract, is an aqueous extract is standardized to 10% salicylic acid that has been shown to possess potent in vitro antimicrobial properties. Efficacy testing has shown that the extract has activity against Staphylococcus aureus and Propionibacterium, two of the skin associated with acne. NAB willow bark extract is a source of salicylic acid-like ingredients and contributes effects similar to those seen from the synthetic salicylic acid, but exhibits reduced irritation. In an embodiment, a product composition or carrier includes a beta-hydroxy acid selected from the group consisting of salicylic acid, acetylsalicylic acid, NAB willow bark extract, and combinations thereof, and salts thereof, including pharmaceutically-acceptable salts, cocrystals thereof, complexes thereof, or derivatives thereof, including prodrugs. In a preferred embodiment, a product composition or carrier includes salicylic acid. In another preferred embodiment, a product composition or carrier includes NAB willow bark extract. Other active ingredients described elsewhere in this specification may also be beta-hydroxy acids and are hereby included as such in the scope of this disclosure.

Active ingredients in the compositions and methods described herein may also include ingredients, such as natural ingredients, that include both alpha-hydroxy acids and beta-hydroxy acids, and salts thereof, including pharmaceutically-acceptable salts, cocrystals thereof, complexes thereof, or derivatives thereof, including prodrugs. In an embodiment, a product composition or carrier includes a natural mixture of lactic acids and fruit acids (e.g. MFA Complex, Barnet Products Corp., Englewood Cliffs, N.J., USA). MFA Complex is a mixture of lactic acid, citric acid, malic acid, green tea, and water. Other active ingredients described elsewhere in this specification may also include both alpha-hydroxy acids and beta-hydroxy acids and are hereby included as such in the scope of this disclosure.

Active ingredients in the compositions and methods described herein may also include antiseptic agents. Suitable topical antiseptic agents include, for example, those described in: Singal, A.; Thami, G. P. Topical antibacterial agents in dermatology, J Dermatol. 2003, 30, 644-48. In an embodiment, a product composition or carrier includes an antiseptic agent selected from the group consisting of benyzlalkonium chloride, cetyl trimethylammonium bromide, cetylpyridinium chloride, benzethonium chloride, chlorhexidine, octenidine, and benzoyl peroxide, and salts thereof, including pharmaceutically-acceptable salts, cocrystals thereof, complexes thereof, or derivatives thereof, including prodrugs. Other active ingredients described elsewhere in this specification may also be antibiotic agents and are hereby included as such in the scope of this disclosure.

Active ingredients in the compositions and methods described herein may also include antibiotic agents. In an embodiment, a product composition or carrier includes an antibiotic agent selected from the group consisting of erythromycin, clarithromycin, sodium sulfacetamide, tetracycline, tetracycline derivatives, neomycin, polymyxin B, pramoxine, mupirocin, bacitracin, silver sulfadiazine, clindamycin, cefuroxime, cefadroxil, loracarbef, retapamulin, sulconazole, sulfamethoxazole, trimethoprim, metronidazole, benzoyl peroxide, salicylic acid, turmerin, curcumin, and combinations thereof, and salts thereof, including pharmaceutically-acceptable salts, cocrystals thereof, complexes thereof, or derivatives thereof, including prodrugs. Suitable antibiotic agents are described in U.S. Patent Publication No. 2013/0280308 A1 and U.S. Pat. Nos. 2,799,620, 2,888,455, 3,475,407, 4,331,803, and 6,521,271, the disclosures of which are incorporated herein by reference. Other active ingredients described elsewhere in this specification may also be antibiotic agents and are hereby included as such in the scope of this disclosure.

Active ingredients in the compositions and methods described herein may also include anti-inflammatory agents. In an embodiment, a product composition or carrier includes an anti-inflammatory agent selected from the group consisting of flurbiprofen, ibuprofen, naproxen, indomethacin, soy isoflavones, sea kelp, silymarin, quercetin, pomegranate extract, niacinamide, gynostemma, glucosamine, ginkgo biloba, green tea extract, grape seed proanthocyanidins, centella asiatica, boswellia serrata, a-bisabolol (also referred to as alpha bisabolol, α-(−)-bisabolol, or levomenol), bisabolol, α-(±)-bisabolol, β-bisabolol, dipotassium glycrrhizinate, beta glucans, 3-O-ethyl ascorbic acid (also known as 3-O-ethyl ascorbyl ether, ethyl ascorbic acid, and vitamin C ethyl), and allantoin, and salts thereof, including pharmaceutically-acceptable salts, cocrystals thereof, complexes thereof, or derivatives thereof, including prodrugs. In an preferred embodiment, a product composition or carrier includes an anti-inflammatory agent selected from the group consisting of bisabolol, bisabolol active plant extracts, bisabolol (bisabolol, zingiber officinale (ginger) root extract), bisabolol and ginger extract, α-bisabolol, α-(−)-bisabolol, levomenol, α-(±)-bisabolol, or β-bisabolol. Bisabolol and related compounds are described in: Russell, K.; Jacob, S. E. Bisabolol, Dermatitis. 2010, 21, 57-58. Other active ingredients described elsewhere in this specification may also be anti-inflammatory agents and are hereby included as such in the scope of this disclosure.

Active ingredients in the compositions and methods described herein may also include antiviral agents. In an embodiment, a product composition or carrier includes an antiviral agent selected from the group consisting of ganciclovir, penciclovir, valaciclovir, acyclovir, famciclovir, lysine, docosanol, and trifluorothymidine, and salts thereof, including pharmaceutically-acceptable salts, cocrystals thereof, complexes thereof, or derivatives thereof, including prodrugs. Other suitable antiviral agents are described in U.S. Patent Publication Nos. 2013/0216590 A1, 2012/0058959 A1, 2006/0058265 A1, and 2005/0032739 A1, and U.S. Pat. Nos. 8,236,768 and 5,585,379, the disclosures of which are incorporated herein by reference. Other active ingredients described elsewhere in this specification may also be antiviral agents and are hereby included as such in the scope of this disclosure.

Active ingredients in the compositions and methods described herein may also include antifungal agents. In an embodiment, a product composition or carrier includes an antifungal agent selected from the group consisting of ketoconazole, econazole, bifonazole, miconazole, clotrimazole, tioconazole, tolnaftate, tolciclate, terbinafine, nystatin, ciclopirox, ciclopirox olamine, undecylenic alkanolamide, benzoic acid, and combinations thereof, and salts thereof, including pharmaceutically-acceptable salts, cocrystals thereof, complexes thereof, or derivatives thereof, including prodrugs. Suitable antifungal agents are described in U.S. Patent Publication Nos. 2011/0008474 A1, 2011/0251146 A1, and 2004/0101538, and U.S. Pat. Nos. 6,075,056, 8,026,238, 8,586,066, 7,553,835, 8,449,926, and 8,586,066, the disclosures of which are incorporated herein by reference. Other active ingredients described elsewhere in this specification may also be antifungal agents and are hereby included as such in the scope of this disclosure.

Active ingredients in the compositions and methods described herein may also include anti-acne agents. In an embodiment, a product composition or carrier includes an anti-acne agent selected from the group consisting of azelaic acid, mandelic acid, salicylic acid, sulfur, colloidal sulfur, resorcinol, and benzoyl peroxide, and salts thereof, including pharmaceutically-acceptable salts, cocrystals thereof, complexes thereof, or derivatives thereof, including prodrugs. Other active ingredients described elsewhere in this specification may also be anti-acne agents and are hereby included as such in the scope of this disclosure.

Active ingredients in the compositions and methods described herein may also include sunscreen agents. Sunscreen agents are also referred to as UV-filter agents. Sunscreen agents that are safe and effective may be used with the compositions and methods described herein, such as those described in: Shaath, N. A. The Encyclopedia of Ultraviolet Filters, 1st Ed. Allured Publishing Corp., 2007. In an embodiment, a product composition or carrier includes a sunscreen agent that blocks UV-B radiation, UV-A radiation, or both UV-B and UV-A radiation. In a preferred embodiment, a sunscreen agent is butylmethoxydibenzoylmethane (BMDBM). Silicone based UV-filters such as polysiloxane-15 (e.g. PARSOL SLX, DSM Nutritional Products) may also be employed as sunscreen agents. Other sunscreen agents that may use used as active ingredients include, but are not limited to, 2-ethylhexyl 2-cyano-3,3-diphenylacrylate (also known as octocrylene), ethyl 2-cyano-3,3-diphenylacrylate, 4-methyl benzylidene camphor, 3-benzylidene camphor, camphor benzalkonium methosulfate, polyacrylamidomethyl benzylidene camphor, sulfabenzylidene camphor, sulphomethyl benzylidene camphor, terephthalylidene dicamphorsulfonic acid (e.g., MEXORYL SX); ethylhexyl methoxycinnamate (e.g., PARSOL MCX), ethoxyethyl methoxycinnamate, isoamyl methoxycinnamate, encapsulated ethylhexyl methoxycinnamate (e.g., EUSOLEX UV-pearls), p-aminobenzoic acid, 2-ethylhexyl p-dimethylaminobenzoate, N-oxypropylenated ethyl p-aminobenzoate, glyceryl p-aminobenzoate, benzophenone-3, benzophenone-4, 2,2′,4,4′-tetrahydroxy-benzophenone, 2,2′-dihydroxy-4,4′-dimethoxybenzophenone, di-(2-ethylhexyl) 4-methoxybenzalmalonate, drometrizole trisiloxane (e.g., MEXORYL XL), 2-phenyl benzimidazole sulfonic acid (e.g., PARSOL HS), isopropylbenzyl salicylate, benzyl salicylate, butyl salicylate, ethylhexyl alicylate (PARSOL EHS), isooctyl salicylate, homomenthyl salicylate (also known as homosalate, e.g. PARSOL HMS), ethylhexyl triazone, diethylhexyl butamido triazone, bis-ethylhexyloxyphenol methoxyphenyl triazine, 2,4,6-tris[1,1-′biphenyl]-4-yl-1,3,5-triazine, 2,2′-methylene-bis-(6-(2H-benzotriazole-2-yl)-4-(1,1,3,3,-tetramethylbutyl)-phenol, 2-(4-diethylamino-2-hydroxy-benzoyl)-benzoic acid hexylester, 2,4-bis-[5-1 (dimethylpropyl)benzoxazol-2-yl-(4-phenyl)-imino]-6-(2-ethylhexyl)-imino-1,3,5-triazin (Uvasorb® K2A); 2,2-(1,4-phenylene)bis-(1H benzimidazol-4,6-disulfonic acid) (Neoheliopan® AP); 1,1′-(1,4-piperazinediyl)bis[1-[4-(diethylamino)-2-hydroxybenzoyl]phenyl]-methanone, bis(butylbenzoate) diaminotriazine aminopropyltrisiloxane, and salts thereof, including pharmaceutically-acceptable salts, cocrystals thereof, complexes thereof, or derivatives thereof, including prodrugs. Other active ingredients described elsewhere in this specification may also be sunscreen agents and are hereby included as such in the scope of this disclosure.

Active ingredients in the compositions and methods described herein may also include skin whitening agents. In an embodiment, a product composition or carrier includes a skin whitening agent selected from the group consisting of L-ascorbic acid, 3-O-ethyl ascorbic acid, ethyl ascorbic acid, ascorbyl tetraisopalmitate (tetrahexyldecyl ascorbate), ascorbyl glucoside, ferulic acid, gamma oryzanol, glucosamine, L-lactic acid, lemon bioferment, niacinamide, pumpkin bioferment, pomegranate extract, sodium ascorbyl phosphate, resveratrol, ethylhexyl resorcinol, hydroquinone, and soy isoflavones, and salts thereof, including pharmaceutically-acceptable salts, cocrystals thereof, complexes thereof, or derivatives thereof, including prodrugs. Suitable skin whitening agents are described in U.S. Patent Publication Nos. 2007/0105947 A1, 2006/0210497 A1, 2006/0210498 A1, 2008/0131382 A1, 2004/0042983 A1, and 2002/0176903 A1, and U.S. Pat. Nos. 4,959,393, 6,132,740, 6,504,037, and 6,682,763, the disclosures of which are incorporated herein by reference. Other active ingredients described elsewhere in this specification may also be skin whitening agents and are included in the scope of this disclosure.

Active ingredients in the compositions and methods described herein may also include plant extracts. In an embodiment, a product composition or carrier includes a plant extract selected from the group consisting of bakuchiol, meroterpenes, terpenophenol-related compounds, zingiber officinale (ginger) root extract, hamamelis virginiana, macademia glycerides, laminaria digitata extract, medicago sativa alfalfa extract, and cichorium intybus (chicory) root extract, and salts thereof, including pharmaceutically-acceptable salts, cocrystals thereof, complexes thereof, or derivatives thereof, including prodrugs. Suitable plant extracts for use with the present compositions and methods are also described in U.S. Patent Publication Nos. 2011/0086116 A1, 2012/0276025 A1, 2013/0243708 A1, 2013/0216635 A1, 2013/0236403 A1, 2013/0309332 A1, 2012/0282195 A1, 2012/0195923 A1, 2010/0316747 A1, 2011/0318437 A1, 2003/0180231 A1, and 2002/0176903 A1, and U.S. Pat. Nos. 8,454,943, 8,609,072, 8,273,387, 8,481,089, 8,444,959, 8,636,989, 8,043,637, 8,313,783, and 6,682,763, the disclosures of which are incorporated herein by reference. Other active ingredients described elsewhere in this specification may also be plant extracts and are hereby included as such in the scope of this disclosure.

Active ingredients in the compositions and methods described herein may also include vitamins. In an embodiment, a product composition or carrier includes a vitamin selected from the group consisting of Vitamin A, Vitamin B1, Vitamin B2, Vitamin B3, Vitamin B5, Vitamin B6, Vitamin B7, Vitamin B9, Vitamin B12, Vitamin C, Vitamin D, Vitamin E, Vitamin K, and combinations thereof, and salts thereof, including pharmaceutically-acceptable salts, cocrystals thereof, complexes thereof, or derivatives thereof, including prodrugs. Other active ingredients described elsewhere in this specification may also be vitamins and are hereby included as such in the scope of this disclosure.

Active ingredients in the compositions and methods described herein may also include corticosteriods. In an embodiment, a product composition or carrier includes a corticosteroid selected from the group consisting of hydrocortisone, hydrocortisone acetate, hydrocortisone aceponate, hydrocortisone buteprate, hydrocortisone 17-butyrate, hydrocortisone 21-butyrate, hydrocortisone valerate, cortisone, cortisone acetate, tixocortol pivalate, prednisolone, methylprednisolone, difluprednate, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, halometasone, alclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate, hydrocortisone-17-butyrate, hydrocortisone-17-aceponate, hydrocortisone-17-buteprate, prednicarbate, loteprednol, fluoromethalone, fluoromethalone acetate, medrysone, rimexolone, and combinations thereof, and salts thereof, including pharmaceutically-acceptable salts, cocrystals thereof, complexes thereof, or derivatives thereof, including prodrugs (such as acetate esters or phosphate esters). Other corticosteriods may also be used in the compositions and methods described herein, such as those described in U.S. Pat. No. 8,546,363 and U.S. Patent Publication Nos. 2009/0215735, 2012/0053161, 2012/0129824, and 20120214776, the disclosures of which are incorporated by reference herein. Other active ingredients described elsewhere in this specification may also be corticosteroids and are hereby included as such in the scope of this disclosure.

Active ingredients in the compositions and methods described herein may also include local anesthetic agents. In an embodiment, a product composition or carrier includes a local anesthetic agent selected from the group consisting of lidocaine, benzocaine, xylocaine, butamben, dibucaine, oxybuprocaine, pramoxine, proparacaine, proxymetacaine, tetracaine and combinations thereof, and salts thereof, including pharmaceutically-acceptable salts, cocrystals thereof, complexes thereof, or derivatives thereof, including prodrugs. Other active ingredients described elsewhere in this specification may also be local anesthetic agents and are hereby included as such in the scope of this disclosure.

Active ingredients in the compositions and methods described herein may also include other bioactive ingredients. In an embodiment, a product composition or carrier includes a bioactive ingredient from the group consisting of diphenhydramine, calamine, doxepin, and combinations thereof, and salts thereof, including pharmaceutically-acceptable salts, cocrystals thereof, complexes thereof, or derivatives thereof, including prodrugs. Other active ingredients described elsewhere in this specification may also be bioactive ingredients and are hereby included as such in the scope of this disclosure.

Active ingredients in the compositions and methods described herein may also include ingredients that improve or alter the consumer perception of a product. Non-limiting examples of such active ingredients include active ingredients that provide for a scent or a color. Scents and aromatherapies are known to exert positive effects on mood (enhancement, balance and well being), stress reduction and relaxation, sleep enhancement, self-confidence, and physical and cognitive performance (e.g. boosting of the immune, respiratory, and circulatory systems). In an embodiment, a product composition or carrier includes an active ingredient selected from the group consisting of neroli oil (also known as oil neroli or citrus aurantium dulcis (orange) flower oil), jasmine oil (also known as jasmine absolute), ylang ylang essential oil, lemon essential oil, lime essential oil, grapefruit essential oil, tangerine essential oil, sweet orange essential oil (also known as orange oil), lavender essential oil, lavendin essential oil, geranium essential oil, rose essential oil, peppermint essential oil, spearmint essential oil, Styrax benzoin resion (also known as Styrax benzoin absolute), vanilla absolute, cinnamon essential oil, clove essential oil, and combinations thereof, and salts thereof, including pharmaceutically-acceptable salts, cocrystals thereof, complexes thereof, or derivatives thereof, including prodrugs. In an embodiment, a product composition or carrier includes an active ingredient selected from the group consisting of food grade dyes and cosmetic dyes. Other active ingredients described elsewhere in this specification may also be ingredients that improve the consumer perception of a product s and are hereby included as such in the scope of this disclosure.

Active ingredients in the compositions and methods described herein may also include pigments. In an embodiment, a product composition or carrier includes a pigment, and salts thereof, including pharmaceutically-acceptable salts, cocrystals thereof, complexes thereof, or derivatives thereof, including prodrugs. Suitable pigments are described, for example, in U.S. Patent Publication No. 2014/0010769 A1, the disclosure of which is incorporated by reference herein. In an embodiment, a method includes the use of a pigment to improve skin tone, skin color, or complexion. In an embodiment, a method includes the use of a product composition or carrier to improve skin tone, skin color, or complexion.

In some embodiments, an active ingredient or compound that is capable of ionization may be employed in the form of its free base or free acid, or may be used in the form of a salt, including a pharmaceutically acceptable salt. Suitable salts and procedures for making salts are known to those of ordinary skill in the art and are described, for example, in P. H. Stahl and C. G. Wermuth, Handbook of Pharmaceutical Salts: Properties, Selection, and Use, Wiley-VCH, Zurich, 2002. In an embodiment, alternative salts of an active ingredient with pharmaceutically acceptable acids may also be utilized, for example salts derived from the functional free base and acids including, but not limited to, hydrochloric acid, palmitic acid, hydrobromic acid, phosphoric acid, acetic acid, fumaric acid, maleic acid, salicylic acid, citric acid, oxalic acid, lactic acid, malic acid, methanesulphonic acid and p-toluene sulphonic acid. As used herein, the term salt also includes salts that retain the desired biological activity of the parent compound and exhibit minimal, if any, undesired toxicological effects. Non-limiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acids, naphthalenedisulfonic acids, and polygalacturonic acid; (b) base addition salts formed with polyvalent metal cations such as zinc, calcium, bismuth, barium, magnesium, aluminum, copper, cobalt, nickel, cadmium, sodium, potassium, and the like, or with an organic cation formed from N,N-dibenzylethylene-diamine, ammonium, or ethylenediamine; or (c) combinations of (a) and (b); e.g., a zinc tannate salt or the like.

All alternative physical forms of an active ingredient or its pharmaceutically acceptable derivatives as described herein, including but not limited to alternative crystalline forms, polymorphs, solvates, amorphous forms, cocrystals, cyclodextrin complexes, crown ether complexes, molecular complexes, and dispersions are also within the scope of this invention, and all references to an active ingredient herein includes all alternative physical forms thereof

The term “pharmaceutically acceptable,” as used herein with respect to an active ingredient, salt, cocrystal, complex, derivative, compound, or composition, refers to a form of the active ingredient, compound, or composition that is generally safe for use in pharmaceutical, cosmetic, or food grade products. In an aspect, the disclosure herein also encompasses pharmaceutically acceptable, hydrates, solvates, stereoisomers, cocrystals, complexes, or amorphous solids of the compounds and compositions embodied herein. For example, the term “pharmaceutically acceptable salt” is to describe a salt form of one or more of the compositions herein. In an embodiment, pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the art.

As set forth above, an active ingredient or composition includes salts, such as pharmaceutically acceptable salts, of the compounds in the active ingredient or composition. In some embodiments, the acids which are used to prepare the acid addition salts of the aforementioned compounds or active ingredients are those which form non-toxic acid addition salts, i.e., salts containing pharmacologically acceptable anions, such as the hydrochloride, hydrobromide, hydroiodide, nitrate, sulfate, bisulfate, phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)] salts, among others.

As set forth above, an active ingredient or composition includes salts, such as pharmaceutically acceptable salts, of the compounds in the active ingredient or composition. In some embodiments, an active ingredient or compound comprises base addition salts of the compounds or active ingredient in the compositions. The chemical bases that may be used as reagents to prepare pharmaceutically acceptable base salts of the present compounds that are acidic in nature are those that form non-toxic base salts with such compounds. Such non-toxic base salts include, but are not limited to those derived from cations such as alkali metal cations (e.g., potassium and sodium) and alkaline earth metal cations (e.g., calcium and magnesium), ammonium or water-soluble amine addition salts such as N-methylglucamine (meglumine), and the alkanolammonium and other base salts of pharmaceutically acceptable organic amines, among others.

Modifications of an active ingredient can affect the solubility, bioavailability and rate of metabolism of the active species, thus providing control over the delivery of the active species. Further, the modifications can affect the activity of the compound, in some cases increasing the activity over the parent compound. This can be assessed by preparing the derivative and testing its activity according to the methods encompassed herein, or other methods known to those skilled in the art.

The term “pharmaceutically acceptable derivative” or “derivative,” as used herein, describes a chemical derivative, such as a pharmaceutically acceptable prodrug form of an active ingredient (e.g. an ester, phosphate ester, or ether) which, upon administration to a patient, provides directly or indirectly the present compound or an active metabolite of an active ingredient. Derivatives and their preparation are known to one of ordinary skill in the art, and are described for example in J. Rautio, H. Kumpulainen, T. Heimbach, R. Oliyai, D. Oh, Dooman; T. Järvinen, and J. Savolainen, Prodrugs: Design and clinical applications, Nat. Rev. Drug Disc., 2008, 7, 255-270.

In an embodiment, the compositions disclosed here may be formulated using other pharmaceutically acceptable carriers. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

In other embodiments, as set forth in greater detail elsewhere herein, the dosage and dosing regimen for the active ingredients may be optimized based on the health and condition of the subject to be treated, as well as the desired outcome of the treatment.

### Dosage Forms and Amounts

In an embodiment, as set forth in detail elsewhere herein, it has now been found that the compositions described herein as administered to a subject provide surprising efficacy for treatment of skin conditions and other disorders. In an embodiment, an active ingredient is administered topically to a subject at a dose of less than 0.01 mg/kg. In an embodiment, an active ingredient is administered topically to a subject at a dose of less than 0.5 mg/kg.

In an embodiment, a dose encompassed herein may be administered as a composition based on the weight of the subject. In an embodiment, a dose may be administered per unit weight of the subject (e.g., mg of a composition described herein per kg weight of subject). In an embodiment, a dose encompassed herein may be administered as a composition based solely on the weight of the dose, without regard to the weight of the subject (e.g., mg of a composition described herein per dose administered to subject). In an embodiment, the dose is determined based on the weight of the active ingredients in the carrier. In another embodiment, the dose is determined based on the total weight of the active ingredients of the composition in the carrier.

In an embodiment, administration of a compound for any purpose as described herein, in any form or combination described herein, may include administering an active ingredient or a pharmaceutically acceptable salt thereof at a dose of 1 ng to 100 μg, 5 ng to 75 μg, 10 ng to 50 μg, 25 ng to 40 μg, 50 ng to 30 μg, 75 ng to 20 μg, 100 ng to 10 μg, 250 ng to 5 μg, 500 ng to 200 μg, 750 ng to 100 μg, 1 μg to 75 μg, 5 μg to 50 μg, or 10 μg to 40 μg. By way of a non-limiting example, and as set forth in detail elsewhere herein, a dose of 10 μg to 40 μg, for example, represents a dosage range of 10 μg per kg of subject weight to 40 μg per kg of subject weight, and can also represent a dosage range of 10 μg administered to a subject to 40 μg administered to a subject. In an embodiment, any of the foregoing active ingredients may be administered at any of the foregoing doses or masses.

In an embodiment, administration of a compound for any purpose as described herein, in any form or combination described herein, may include administering an active ingredient or a salt, cocrystal, complex, or derivative thereof, e.g. a pharmaceutically acceptable salt, at a dose of 1 ng to 1 g, 5 ng to 1 g, 10 ng to 1 g, 25 ng to 1 g, 50 ng to 1 g, 75 ng to 1 g, 100 ng to 750 mg, 500 ng to 500 mg, 10 μg to 200 mg, 15 μg to 190 mg, 25 μg to 180 mg, 50 μg to 170 mg, 75 μg to 160 mg, 100 μg to 150 mg, 250 μg to 140 mg, 400 μg to 130 mg, 500 μg to 128 mg, 600 μg to 100 mg, 750 μg to 75 mg, 900 μg to 50 mg, or 0.1 mg to 64 mg. In an embodiment, any of the foregoing active ingredients may be administered at any of the foregoing doses or masses.

In an embodiment, an active ingredient or a salt, cocrystal, complex, or derivative thereof, e.g. a pharmaceutically acceptable salt, may be administered to a subject according to the compositions and methods encompassed herein at a dose of about 1000 mg or less, about 500 mg or less, about 200 mg or less, about 150 mg or less, about 100 mg or less, about 50 mg or less, about 40 mg or less, about 30 mg or less, about 20 mg or less, about 10 mg or less, about 9 mg or less, about 8 mg or less, about 7 mg or less, about 6 mg or less, about 5 mg or less, about 4 mg or less, about 3 mg or less, about 2 mg or less, about 1 mg or less, about 0.9 mg or less, about 0.8 mg or less, about 0.7 mg or less, about 0.6 mg or less, about 0.5 mg or less, about 0.4 mg or less, about 0.3 mg or less, about 0.2 mg or less, about 0.1 mg or less, about 0.09 mg or less, about 0.08 mg or less, about 0.07 mg or less, about 0.06 mg or less, about 0.05 mg or less, about 0.04 mg or less, about 0.03 mg or less, about 0.02 mg or less, about 0.01 mg or less, about 0.009 mg or less, about 0.008 mg or less, about 0.007 mg or less, about 0.006 mg or less, about 0.005 mg or less, about 0.004 mg or less, about 0.003 mg or less, about 0.002 mg or less, about 0.001 mg or less, or about 0.0005 mg or less. In an embodiment, any of the foregoing active ingredients may be administered at any of the foregoing doses or masses.

In an embodiment, an active ingredient or a salt, cocrystal, complex, or derivative thereof, e.g. a pharmaceutically acceptable salt, may be administered to a subject according to the compositions and methods encompassed herein at a dose of about 1 g, about 500 mg or more, about 200 mg or more, about 150 mg or more, about 100 mg or more, about 50 mg or more, about 40 mg or more, about 30 mg or more, about 20 mg or more, about 10 mg or more, about 9 mg or more, about 8 mg or more, about 7 mg or more, about 6 mg or more, about 5 mg or more, about 4 mg or more, about 3 mg or more, about 2 mg or more, about 1 mg or more, about 0.9 mg or more, about 0.8 mg or more, about 0.7 mg or more, about 0.6 mg or more, about 0.5 mg or more, about 0.4 mg or more, about 0.3 mg or more, about 0.2 mg or more, about 0.1 mg or more, about 0.09 mg or more, about 0.08 mg or more, about 0.07 mg or more, about 0.06 mg or more, about 0.05 mg or more, about 0.04 mg or more, about 0.03 mg or more, about 0.02 mg or more, about 0.01 mg or more, about 0.009 mg or more, about 0.008 mg or more, about 0.007 mg or more, about 0.006 mg or more, about 0.005 mg or more, about 0.004 mg or more, about 0.003 mg or more, about 0.002 mg or more, about 0.001 mg or more, or about 0.0005 mg or more. In an embodiment, any of the foregoing active ingredients may be administered at any of the foregoing doses or masses.

In an embodiment, an active ingredient or a salt, cocrystal, complex, or derivative thereof, e.g. a pharmaceutically acceptable salt, may be administered to a subject according to the compositions and methods encompassed herein at a dose of about 1000 mg, about 500 mg, about 200 mg, about 150 mg, about 100 mg, about 50 mg, about 40 mg, about 30 mg, about 20 mg, about 10 mg, about 9 mg, about 8 mg, about 7 mg, about 6 mg, about 5 mg, about 4 mg, about 3 mg, about 2 mg, about 1 mg, about 0.9 mg, about 0.8 mg, about 0.7 mg, about 0.6 mg, about 0.5 mg, about 0.4 mg, about 0.3 mg, about 0.2 mg, about 0.1 mg, about 0.09 mg, about 0.08 mg, about 0.07 mg, about 0.06 mg, about 0.05 mg, about 0.04 mg, about 0.03 mg, about 0.02 mg, about 0.01 mg, about 0.009 mg, about 0.008 mg, about 0.007 mg, about 0.006 mg, about 0.005 mg, about 0.004 mg, about 0.003 mg, about 0.002 mg, about 0.001 mg, or about 0.0005 mg. In an embodiment, any of the foregoing active ingredients may be administered at any of the foregoing doses or masses.

In an embodiment, an active ingredient or a salt, cocrystal, complex, or derivative thereof, e.g. a pharmaceutically acceptable salt, may be administered to a subject according to the compositions and methods encompassed herein at a dose of 1000 mg, 500 mg, 200 mg, 150 mg, 100 mg, 50 mg, 40 mg, 30 mg, 20 mg, 10 mg, 9 mg, 8 mg, 7 mg, 6 mg, 5 mg, 4 mg, 3 mg, 2 mg, 1.9 mg, 1.8 mg, 1.7 mg, 1.6 mg, 1.5 mg, 1.4 mg, 1.3 mg, 1.2 mg, 1.1 mg, 1 mg, 0.9 mg, 0.8 mg, 0.7 mg, 0.6 mg, 0.5 mg, 0.4 mg, 0.3 mg, 0.2 mg, 0.1 mg, 0.09 mg, 0.08 mg, 0.07 mg, 0.06 mg, 0.05 mg, 0.04 mg, 0.03 mg, 0.02 mg, 0.01 mg, 0.009 mg, 0.008 mg, 0.007 mg, 0.006 mg, 0.005 mg, 0.004 mg, 0.003 mg, 0.002 mg, 0.001 mg, or 0.0005 mg. In an embodiment, any of the foregoing active ingredients may be administered at any of the foregoing doses or masses.

In an embodiment, an active ingredient or a salt, cocrystal, complex, or derivative thereof, e.g. a pharmaceutically acceptable salt, may be administered to a subject according to the compositions and methods encompassed herein at a dose of about 1000 mg/kg, about 500 mg/kg, about 200 mg/kg, about 150 mg/kg, about 100 mg/kg, about 50 mg/kg, about 40 mg/kg, about 30 mg/kg, about 20 mg/kg, about 10 mg/kg, about 9 mg/kg, about 8 mg/kg, about 7 mg/kg, about 6 mg/kg, about 5 mg/kg, about 4 mg/kg, about 3 mg/kg, about 2 mg/kg, about 1.9 mg/kg, about 1.8 mg/kg, about 1.7 mg/kg, about 1.6 mg/kg, about 1.5 mg/kg, about 1.4 mg/kg, about 1.3 mg/kg, about 1.2 mg/kg, about 1.1 mg/kg, about 1 mg/kg, about 0.9 mg/kg, about 0.8 mg/kg, about 0.7 mg/kg, about 0.6 mg/kg, about 0.5 mg/kg, about 0.4 mg/kg, about 0.3 mg/kg, about 0.2 mg/kg, about 0.1 mg/kg, about 0.09 mg/kg, about 0.08 mg/kg, about 0.07 mg/kg, about 0.06 mg, about 0.05 mg, about 0.04 mg, about 0.03 mg/kg, about 0.02 mg, about 0.01 mg, about 0.009 mg, about 0.008 mg, about 0.007 mg, about 0.006 mg/kg, about 0.005 mg/kg, about 0.004 mg/kg, about 0.003 mg/kg, about 0.002 mg/kg, about 0.0019 mg/kg, about 0.0018 mg/kg, about 0.0017 mg/kg, about 0.0016 mg/kg, about 0.0015 mg/kg, about 0.0014 mg/kg, about 0.0013 mg/kg, about 0.0012 mg/kg, about 0.0011 mg/kg, about 0.001 mg/kg, about 0.0005 mg/kg, or any range determinable from the preceding dosages (e.g., about 0.0013 mg/kg to about 0.0016 mg/kg or about 0.001 mg/kg to about 0.002 mg/kg). In an embodiment, any of the foregoing active ingredients may be administered at any of the foregoing doses, mass ratios, or concentrations.

In an aspect, a method of administering an active ingredient or a salt, cocrystal, complex, or derivative thereof, e.g. a pharmaceutically acceptable salt, may include titration of the compound up to a predetermined level. In one embodiment, an active ingredient is used at a specified level (e.g., 0.05 mg b.i.d., 0.1 mg b.i.d., 0.2 mg b.i.d., 0.4 mg b.i.d., 0.6 mg b.i.d., 0.8 mg b.i.d., 1 mg b.i.d., 2 mg b.i.d., 4 mg b.i.d., 8 mg b.i.d., 16 mg b.i.d., 32 mg b.i.d., 64 mg b.i.d.). In one embodiment, an active ingredient is used at a specified level (e.g., about 0.05 mg b.i.d., about 0.1 mg b.i.d., about 0.2 mg b.i.d., about 0.4 mg b.i.d., about 0.6 mg b.i.d., about 0.8 mg b.i.d., about 1 mg b.i.d., about 2 mg b.i.d., about 4 mg b.i.d., about 8 mg b.i.d., about 16 mg b.i.d., 32 mg b.i.d., 64 mg b.i.d.). In one embodiment, an active ingredient is titrated up to a predetermined dosage (e.g., titration up to 0.1 mg b.i.d., 0.2 mg b.i.d., 0.4 mg b.i.d., 0.6 mg b.i.d., 0.8 mg b.i.d., 1 mg b.i.d., 2 mg b.i.d., 4 mg b.i.d., 8 mg b.i.d., 16 mg b.i.d., 32 mg b.i.d., 64 mg b.i.d., etc. . . . ). In one embodiment, an active ingredient is titrated up to a predetermined dosage (e.g., titration up to about 0.1 mg b.i.d., about 0.2 mg b.i.d., about 0.4 mg b.i.d., about 0.6 mg b.i.d., about 0.8 mg b.i.d., about 1 mg b.i.d., about 2 mg b.i.d., about 4 mg b.i.d., about 8 mg b.i.d., about 16 mg b.i.d., about 32 mg b.i.d., about 64 mg b.i.d., etc. . . . ). In an embodiment, an active ingredient is titrated up to a predetermined dosage as the dosage is described elsewhere herein.

In an embodiment, an active ingredient or a salt, cocrystal, complex, or derivative thereof, e.g. a pharmaceutically acceptable salt, may be administered to a subject according to the compositions and methods encompassed herein at a dose that is achieved by a concentration in a product composition. In an embodiment, a composition or product composition includes an active ingredient at a concentration of 0.001%, 0.005%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, or 10.0% by weight. In an embodiment, a composition or product composition includes an active ingredient at a concentration of 0.001%, 0.005%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, or 10.0% by volume. In an embodiment, a composition or product composition includes an active ingredient at a concentration of 0.001% to 0.005%, 0.0005% to 0.01%, 0.01% to 0.05%, 0.01% to 0.1%, 0.1% to 0.3%, 0.1% to 1.0%, 0.1% to 3%, 1% to 3%, 1% to 5%, or 1% to 10% by weight. In an embodiment, a composition or product composition includes an active ingredient at a concentration of 0.001% to 0.005%, 0.0005% to 0.01%, 0.01% to 0.05%, 0.01% to 0.1%, 0.1% to 0.3%, 0.1% to 1.0%, 0.1% to 3%, 1% to 3%, 1% to 5%, or 1% to 10% by volume. In an embodiment, any of the foregoing active ingredients may be administered at any of the foregoing concentration or mass percentages.

In an embodiment, an active ingredient or a salt, cocrystal, complex, or derivative thereof, e.g. a pharmaceutically acceptable salt, may be used and/or administered to a subject based a desired plasma concentration of the active ingredient. In an embodiment, the dosage of active ingredient administered to a subject is determined by identifying the dosage required to obtain a plasma concentration of about 10 ng/ml of the active ingredient in a subject. In an embodiment, the dosage of active ingredient administered to a subject is determined by identifying the dosage required to obtain a plasma concentration of about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 12 ng/ml, about 14 ng/ml, about 16 ng/ml, about 18 ng/ml, about 20 ng/ml, about 25 ng/ml, about 30 ng/ml, about 35 ng/ml, about 40 ng/ml, about 45 ng/ml, or about 50 ng/ml of the active ingredient in a subject. In an embodiment, any of the foregoing active ingredients may be administered at any of the foregoing doses or concentrations.

The dose may be administered as a weekly dose, a single daily dose, twice daily (b.i.d.), three times daily, four times daily, five times daily, or more frequently. In an embodiment, administration frequency may be between 1 and 5 times a day. In another embodiment, administration frequency may be between 2 and 4 times a day. In another embodiment, administration frequency may be at least 3 times a day. In another embodiment, administration frequency may be twice a day. In another embodiment, administration frequency may be once a day. In another embodiment, administration frequency may be less frequent than once a day. In other embodiments, administration frequency may be once every 2 days or once every 3 days or once every 4 days or once every 5 days or once every 6 days. In another embodiment, administration frequency may be once a week. In another embodiment, administration frequency may be on demand, as therapeutic treatment is required or desired. It will be understood, based on the disclosure encompassed herein, how to determine whether a subject needs an additional and/or continued dose. It will also be understood that the selected dosing frequency may require an adjustment of the dosage of active ingredient. It will also be understood, based on the disclosure encompassed herein, that the selected dosage of active ingredient may require an adjustment of the dosing frequency. The disclosure encompassed herein, in combination with the skill in the art, will enable the skilled artisan to optimize both the dosage of the active ingredient and the frequency of administration of the active ingredient to treat a subject in need thereof.

It will further be understood by the skilled artisan that, in addition to the above embodiments of dosage and dosing regimens, both the dosage and the dosing regimen will be considered and each adjusted, as necessary, in view of the condition of the subject being treated.

### Co-Administration of Compositions

In an embodiment, a composition described herein is administered in conjunction with one or more other medications or consumer products. Such other medications or consumer products may be administered or co-administered in forms and dosages as known in the art, or in the alternative, as has been described above for administration of active ingredients using the compositions described herein.

The term “co-administration” or “combination therapy” is used to describe a therapy in which at least two compositions, which may include one or more product compositions as described herein, are used to treat, address, or affect a skin condition or another disorder as described herein at the same time. In an embodiment, at least two compositions in effective amounts are used to treat, address, or affect a skin condition or another disorder as described herein at the same time. In another embodiment, at least two active ingredients, the combination of which comprises an effective amount, are used to treat, address, or affect a skin condition or another disorder as described herein at the same time. In an embodiment, the result of treatment with the at least two compositions may be additive of the treatment results obtained using each composition separately, either directly additive, or additive to a degree lesser than the results obtained with the two compositions separately. In an embodiment, the result of treatment with the at least two compositions may be synergistic, to varying degrees. In an embodiment, the result of treatment with the at least two compositions may be greater than the treatment results obtained using each composition separately. In an aspect, the result of treatment for at least two active ingredients is less than that obtained with the active ingredients separately, while the other active ingredients in the composition are about the same as the results of treatment obtained separately. In an aspect, the result of treatment for all active ingredients in the composition is less than that obtained with the active ingredients separately.

Although the term co-administration encompasses the administration of two compositions to the patient at the same time, it is not necessary that the compositions be administered to the patient at the same time, although effective amounts of the individual active ingredients delivered by the compositions will be present in the patient at the same time.

A product composition described herein may advantageously be administered in combination with at least one other therapeutic agent to provide improved treatment of a skin condition or another disorder. The combinations, uses and methods of treatment of the invention may also provide advantages in treatment of patients or consumers who fail to respond adequately to other known treatments. In an embodiment, a product composition described herein may be administered to a patient already undergoing treatment with at least one other skin care composition, to provide improved treatment of any combination of conditions described herein. In an embodiment, a product composition set forth herein is co-administered with one or more lotions, foams, or creams.

### Methods of Treatment and Methods of Skin Care

The product compositions described herein are useful and effective for skin care and for the treatment of a variety of skin conditions and disorders. For example, the product compositions may be used to improve skin hydration, improve skin firmness, improve skin elasticity, reduce the mean area of skin wrinkles, reduce pore size, improve skin clarity, and reduce skin blotchiness, or give the cosmetic impression of any of the foregoing. In an embodiment, a product composition described herein is used for treating a skin condition or disorder. In an embodiment, a method of treating a skin condition or disorder using a product composition described herein comprises the step of administering the composition to a subject. In an embodiment, a method of treating a skin condition or disorder using a product composition described herein comprises the step of topically administering the composition. In an embodiment, a method of treating a skin condition or disorder using a product composition described herein comprises the step of topically administering the composition to the facial skin of a human subject.

The skin conditions that may be treated or cosmetically addressed by the product compositions include, but are not limited to, dry skin, eczema, skin discoloration, ashen skin, acne, acne vulgaris, skin blemishes, skin discoloration, skin wrinkles, psoriasis, diaper rash, sun burn, seborrhea, atopic dermatitis, lichen simplex chronicus, poison ivy, inflammatory pruritus, photo-aging, smoker's lines, thin skin, elastosis, freckles, solar lentigo, guttate hypomelanosis, ideopathic guttate hypomelanosis, white marks, telangiectases, cherry angiomas, senile purpura, solar comedones, colloid milia, and seborrhoeic keratoses. Various skin conditions that may be treated, prevented, or cosmetically addressed using the product compositions described herein are also described in U.S. Patent Publication Nos. 2002/0004056 A1, 2004/0156805, 2006/0257845, and 2013/0210759, the disclosure of which is incorporated herein by reference.

The compositions described herein are also effective in the topical delivery of active ingredients, including cosmetic ingredients, ingredients that improve the appearance of skin, pharmaceutical active ingredients used to treat or prevent a disorder or condition, and ingredients used to improve consumer perception of a product. In an embodiment, a method of treating a disease or illness in a mammal includes the step of delivering a pharmaceutical active ingredient using a composition described herein. In an embodiment, a method of improving the appearance of skin includes the step of delivering a cosmetic ingredient using a composition described herein. In an embodiment, a method of improving the consumer perception of a product includes the step of delivering a cosmetic ingredient using a composition described herein.

The compositions described herein are effective in treating skin conditions and disordered when administered over various periods. In an embodiment, a composition described herein is administered once a day for a period of 1, 2, 3, 4, 5, 6, or 7 days. In an embodiment, a composition described herein is administered twice a day for a period of 1, 2, 3, 4, 5, 6, or 7 days. In an embodiment, a composition described herein is administered three times a day for a period of 1, 2, 3, 4, 5, 6, or 7 days. In an embodiment, a composition described herein is administered once a day for a period of 1, 2, 3, 4, 5, 6, 7, or 8 weeks, or for a period of 1 month, 2 months, 3 months, 6 months, or 1 year. In an embodiment, a composition described herein is administered twice a day for a period of 1, 2, 3, 4, 5, 6, 7, or 8 weeks, or for a period of 1 month, 2 months, 3 months, 6 months, or 1 year. In an embodiment, a composition described herein is administered three times a day for a period of 1, 2, 3, 4, 5, 6, 7, or 8 weeks, or for a period of 1 month, 2 months, 3 months, 6 months, or 1 year.

The term “treating” (and corresponding terms “treat” and “treatment”) includes palliative, restorative, and preventative (“prophylactic”) treating of a subject, as well as cosmetic applications that provide the appearance of treatment. The term “palliative treating” refers to treatment that eases or reduces the effect or intensity of a condition in a subject without curing the condition. The term “preventative treating” (and the corresponding term “prophylactic treating”) refers to treatment that prevents the occurrence of a condition in a subject. The term “restorative treating” (“curative”) refers to treatment that halts the progression of, reduces the pathologic manifestations of, or entirely eliminates a condition in a subject. Treating can be done with a therapeutically effective amount of compound, salt or composition that elicits the biological or medicinal response of a tissue, system or subject that is being sought by an individual such as a researcher, doctor, veterinarian, or clinician. The term “treatment” will also be understood to include not only a complete remission of all symptoms experienced by the treated individual, but also the alleviation of one or more existing symptoms, as well as the prevention of occurrence of symptoms by preemptive administration of a composition described herein to an individual prone to or likely to develop skin conditions. The methods disclosed herein can be used for treatment of any mammal exhibiting symptoms of a skin condition, e.g., for treatment of mammals, such as cats, dogs, rats, rabbits, horses and the like; however, in a preferred embodiment, the method is used to treat humans.

In an embodiment, skin firmness is improved by at least 5%. In an embodiment, skin firmness is improved by at least 10%. In an embodiment, skin firmness is improved by between 1% and 25%. In an embodiment, skin hydration is improved by at least 5%. In an embodiment, skin hydration is improved by at least 10%. In an embodiment, skin hydration is improved by between 1% and 25%. In an embodiment, mean fine line area is improved by at least 5%. In an embodiment, mean fine line area is improved by at least 10%. In an embodiment, mean fine line area is improved by between 1% and 25%. In an embodiment, mean area of skin wrinkles is improved by at least 5%. In an embodiment, mean area of skin wrinkles is improved by at least 10%. In an embodiment, mean area of skin wrinkles is improved by between 1% and 25%. In an embodiment, mean skin clarity is improved by at least 5%. In an embodiment, mean skin clarity is improved by at least 10%. In an embodiment, mean skin clarity is improved by between 1% and 25%.

### Examples

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Skin Care Product Composition

A product composition within the ranges or with the quantities specified in Formula A in Table 1 is prepared by the following procedure, and may also be prepared using alternative mixing, blending, homogenizing, spraying, or related manufacturing procedures known to those of ordinary skill in the art,

TABLE 1
Formula A

| | Amount | |
|---|---|---|
| Component (TRADE NAME) | (% w/w) | Primary Purpose |
| Dimethicone and trisiloxane | 30 to 80 | Carrier |
| (XIAMETER PMX-1184 | | |
| silicone oil) | | |
| SD alcohol 40B, 200 proof | 10 to 30 | Carrier |
| Diisopropyl sebacate (DUB DIS) | 1 to 10 | Carrier |
| 3-O-ethyl ascorbic acid | 1 to 1.5 | Anti-aging |
| Bakuchiol (SYTENOL A) | 1 | Retinoid |
| Mandelic acid (ORISTAR MA) | 0.1 to 1 | Anti-aging, anti acne, |
| | | and anti-blemish |
| Niacinamide | 0.1 to 0.3 | Blemish and skin |
| | | healing |
| Azelaic acid (ORISTAR AZA) | 0.1 to 0.5 | Anti-aging, anti acne, |
| | | and anti-blemish |
| Bisabolol, zingiber officinale | 1 | Skin calming |
| (ginger) | | |
| root extract (SYMRELIEF 100) | | |
| Dimethyl isosorbide with 10% | 1 | Anti-aging, treatment |
| hydroxypinacolone retinoate | | for fine lines |
| (GRANACTIVE RETINOID) | | |
| Caprylic/capric triglyceride, | 1 | Skin brightening |
| hexylresorcinol, ethyl linoleate | | |
| (ASYNTRA SL) | | |
| Blended Medicago Sativa Alfalfa | 0.001 to 2 | Anti-aging |
| extract and Cichorium | | |
| Intybus (Chicory) | | |
| root extract (PHYTINOL) | | |
| Citrus Aurantium Amara (Bitter | 0.001 to 0.05 | Scent |
| Orange) Oil | | |

The composition is prepared in a stainless steel drum or tank using an air mixer. Tanks with electrical components must not be used as they may pose a risk of explosion. Grounding wires should be employed to ground the drum, air mixer and pump, and operators should wear anti-static straps while dispensing and handling alcohol. To a sanitized, jacketed stainless steel tank equipped with an air mixer, the following materials are sequentially added with mixing: SD Alcohol 40-B, a mixture of caprylic/capric triglyceride, hexylresorcinol, and ethyl linoleate (ASYNTRA SL), a mixture of bisabolol and hydroxymethoxyphenyl decanone (SYMRELIEF 100, Innovadex LLC, Overland Park, Kans., USA), bakuchiol (SYTENOL A), 3-O-ethyl ascorbyl acid (Arg-Enb-Vee, CAS #86404-04-8), azelaic acid (ORISTAR AZA), mandelic acid (ORISTAR MA), and niacinamide PC. Mixing is continued until all solids are dissolved and a uniform mixture is obtained. This mixture is referred to as the “Main Phase.”

In a sanitized, jacketed stainless steel tank equipped with a mixer and scraper, silicone oil (XIAMETER PMX-1184 SIL Fluid, Dow Corning) and diisopropyl sebacate (DUB DIS, Stearinerie Dubois, Cedex, France) are mixed with moderate axial and side sweep mixing. This mixture is referred to as the “Premix Phase.”

The Premix Phase is transferred into the Main Phase and mixed with moderate axial and side sweep mixing until a uniform mixture is obtained. To this combined mixture is added 10% hydroxypinacolone retinoate in dimethyl isosorbide (GRANACTIVE RETINOID, Grant Industries, Elmwood Park, N.J.), blended Medicago Sativa Alfalfa extract and Cichorium Intybus (Chicory) root extract (PHYTINOL, Arbonne, Inc.), and Oil Neroli (also known as neroli oil or by the INCI name of citrus aurantium dulcis (orange) flower oil), which are mixed with moderate axial and side sweep mixing until a uniform mixture is obtained.

### Example 2: Skin Care Product Composition

A product composition is prepared with the ranges specified in Formula B in Table 2.

TABLE 2
Formula B

| Component (TRADE NAME) | Amount (% w/w) | Primary Purpose |
|---|---|---|
| Trisiloxane | 36 | Carrier |
| Dimethicone | 28 | Carrier |
| SD alcohol 40B, 200 proof | 25 | Carrier |
| Diisopropyl sebacate (DUB DIS) | 5 | Carrier |
| Active ingredients | 6 | Retinoid compounds, alpha- |
| | | hydroxy acids, beta-hydroxy acids, |
| | | antiseptic agents, antibiotic agents, |
| | | anti-inflammatory agents, antiviral |
| | | agents, antifungal agents, anti-acne |
| | | agents, sunscreen agents, plant |
| | | extracts, vitamins, corticosteriods, |
| | | local anesthetics, other bioactive |
| | | ingredients, ingredients that |
| | | improve consumer perception, |
| | | pigments, etc. . . |

The composition is prepared using the process described in Example 1. Additional active ingredients may be added as described in Example 1.

### Example 3: Alternative Skin Care Product Composition

A product composition is prepared with the composition specified in Formula C in Table 3.

TABLE 3
Formula C

| Component (TRADE NAME and | Amount | |
|---|---|---|
| Supplier, if applicable) | (% w/w) | Primary Purpose |
| Diisopropyl sebacate (DUB DIS) | 58.55 | Carrier |
| Trisiloxane and dimethicone | 20.00 | Carrier |
| (XIAMETER PMX-1184 SILICONE | | |
| FLUID, Dow Corning) | | |
| SD Alcohol 40-B, 200 Proof (Remet) | 11.00 | Carrier |
| Tetrahexyldecyl ascorbate (BV-OSC, | 0.50 | Anti-aging |
| DD Chem Co/Barnet) | | |
| Cap/cap triglycerides and | 1.00 | Skin brightening |
| hexylresorcinol and ethyl linoleate | | |
| (ASYNTRA SL, Syntheon) | | |
| Bakuchiol (SYSTENOL A, Syntheon) | 1.00 | Retinoid |
| PEG 16 macademia glycerides and | 5.00 | |
| ethanol (DERM X, JRX) | | |
| Azelaic acid (Orient Star) | 0.10 | Anti-aging, anti acne, |
| | | and anti-blemish |
| Mandelic acid (Orient Star) | 0.50 | Anti-aging, anti acne, |
| | | and anti-blemish |
| Niacinamide (DSM) | 0.10 | Blemish and skin |
| | | healing |
| Bisabolol, zingiber officinale (ginger) | 1.00 | Skin calming |
| root extract (SYMRELIEF 100, | | |
| Symrise) | | |
| Phenylethyl resorcinol (SYMWHITE | 0.20 | |
| 377, Symrise) | | |
| Citrus Aurantium Amara (Bitter | 0.05 | Scent |
| Orange) Oil (Alpha Research and | | |
| Development) | | |
| Dimethyl isosorbide with 10% | 1.00 | Anti-aging, treatment |
| hydroxypinacolone retinoate | | for fine lines |
| (GRANACTIVE RETINOID, Grant | | |
| Industries) | | |

The composition is prepared by pre-mixing SD Alcohol 40-B, azelaic acid, mandelic acid, niacinamide, SYMWHITE 377, and DERM X, and heating to 45-50° C. until all solids are dissolved. To this composition is then added a pre-mixed composition of DUB DIS, XIAMETER PMX-1184, BV-OSC, ASYNTRA SL, SYSTENOL A, AND SYMRELIEF 100. Additional active ingredients may also be added.

### Example 4: Aqueous Skin Care Product Composition

A product composition is prepared with the composition specified in Formula D in Table 4.

TABLE 4
Formula D

| Component (TRADE NAME and | Amount | |
|---|---|---|
| Supplier, if applicable) | (% w/w) | Primary Purpose |
| Water | 52.30 | Carrier |
| Pentylene Glycol HYDROLITE 5 | 5.00 | Carrier |
| 616751 (Symrise) | | |
| Propanediol ZEMEA (Dupont, Tate & | 5.00 | Carrier |
| Lyle) | | |
| PEG 16 macademia glycerides and | 5.00 | |
| ethanol (DERM X, JRX) | | |
| Azelaic acid Azelaic acid (Orient Star) | 0.10 | Anti-aging, anti acne, |
| | | and anti-blemish |
| Mandelic acid (Orient Star) | 0.50 | Anti-aging, anti acne, |
| | | and anti-blemish |
| Niacinamide PC (DSM) | 0.20 | Blemish and skin |
| | | healing |
| Water, Laminaria Digitata Extract | 0.50 | |
| MITOSTIME (Barnet) | | |
| Matrixyl syn 6 (Croda) | 2.00 | |
| Phytinol (Silab) | 5.00 | |
| Isoceteth-20 BRIJ IC20-70-LQ-(AP) | 1.50 | |
| (Croda)/“Formerly” ARLASOLVE | | |
| 200 L (Uniq./Croda) | | |
| Tetrahexyldecyl Ascorbate BV-OSC | 0.10 | |
| (DD ChemCo/Barnet) *GRC | | |
| Polysorbate 20 TWEEN 20-LQ-(AP) | 1.50 | |
| (Croda) (Formerly Tween 20) | | |
| Hamamelis Virginiana (Witch Hazel) | 20.00 | |
| Water, 14% Alcohol (Univar) | | |
| Bisabolol, zingiber officinale (ginger) | 0.10 | Skin calming |
| root extract (SYMRELIEF 100, | | |
| Symrise) | | |
| Ethylhexylglycerin SENSIVA SC-50 | 1.00 | |
| (Ross/S&M) | | |
| Phenylethyl resorcinol SYM WHITE | 0.20 | |
| 377 (Symrise) | | |

The composition is prepared by pre-mixing glycerine, HYDROLITE 5, ZEMEA, DERM X, AZA, MA, SYNOVEA HR, SYMWHITE 377, and Niacinamide. The mixture is heated to 40-50° C. until all solids are dissolved. To this is added a pre-mixture of Tween 20, Brij IC20, SYSTENOL A, syn DOI, and SYMRELIEF 100, and SENSIVA SC50. Additional active ingredients may also be added.

### Example 5: Skin Care Product Composition

A product composition is prepared with the compositions or with the ranges specified in Formula E in Table 5.

TABLE 5
Formula E

| Component (TRADE NAME and | Amount | |
|---|---|---|
| Supplier, if applicable) | (% w/w) | Primary Purpose |
| Diisopropyl sebacate (DUB DIS) | 1 to 10 | Carrier |
| Trisiloxane and dimethicone | 40 to 70 | Carrier |
| (XIAMETER PMX-1184 SILICONE | | |
| FLUID, Dow Corning) | | |
| SD Alcohol 40-B, 200 Proof (Remet) | 10 to 30 | Carrier |
| 3-O-ethyl ascorbic acid | 0.5 to 2.0 | Anti-aging |
| Cap/cap triglycerides and | 0.5 to 2.0 | Skin brightening |
| hexylresorcinol and ethyl linoleate | | |
| (ASYNTRA SL, Syntheon) | | |
| Bakuchiol (SYSTENOL A, Syntheon) | 0.5 to 2.0 | Retinoid |
| Azelaic acid (Orient Star) | 0.05 to 0.5 | Anti-aging, anti acne, |
| | | and anti-blemish |
| Mandelic acid (Orient Star) | 0.1 to 1.0 | Anti-aging, anti acne, |
| | | and anti-blemish |
| Niacinamide (DSM) | 0.05 to 0.5 | Blemish and skin |
| | | healing |
| Bisabolol, zingiber officinale (ginger) | 0.5 to 1.2 | Skin calming |
| root extract (SYMRELIEF 100, | | |
| Symrise) | | |
| Citrus Aurantium Amara (Bitter | 0.01 to 0.1 | Scent |
| Orange) Oil (Alpha Research and | | |
| Development) | | |
| Dimethyl isosorbide with 10% | 0.7 to 1.1 | Anti-aging, treatment |
| hydroxypinacolone retinoate | | for fine lines |
| (GRANACTIVE RETINOID, Grant | | |
| Industries) | | |
| PHYTINOL | 0.001 to 1 | |

The composition may be prepared using the process described in Example 1.

Additional active ingredients may be added as desired, and the levels of active ingredients may be varied.

### Example 6: Skin Care Product Composition with a Drier, Less Silky Feel

A product composition is prepared with the composition specified in Formula F in Table 6. This composition provides for a drier, less silky feel when applied, which may be advantageous in some products and methods of use.

TABLE 6
Formula F

| Component (TRADE NAME) | Amount (% w/w) | Primary Purpose |
|---|---|---|
| Trisiloxane | 36% | Carrier |
| Ethyl trisiloxane | 8% | Carrier |
| Dimethicone | 20% | Carrier |
| SD alcohol 40B, 200 proof | 25% | Carrier |
| Diisopropyl sebacate (DUB | 5% | Carrier |
| DIS) | | |
| Active ingredients | 6% | Retinoid compounds, alpha-hydroxy |
| | | acids, beta-hydroxy acids, antiseptic |
| | | agents, antibiotic agents, anti- |
| | | inflammatory agents, antiviral agents, |
| | | antifungal agents, anti-acne agents, |
| | | sunscreen agents, plant extracts, |
| | | vitamins, corticosteriods, local |
| | | anesthetics, other bioactive |
| | | ingredients, ingredients that improve |
| | | consumer perception, pigments, etc. . . |

The composition may be prepared using the process described in Example 1. Additional active ingredients may be added as desired.

### Example 7: Skin Care Product Composition with a Wet Feel

A product composition is prepared with the composition specified in Formula G in Table 7. This composition provides for a wet feel, which may be advantageous in some products and methods of use.

TABLE 7
Formula G

| Component (TRADE NAME) | Amount (% w/w) | Primary Purpose |
|---|---|---|
| Trisiloxane | 30 | Carrier |
| Dimethicone | 34 | Carrier |
| SD alcohol 40B, 200 proof | 25 | Carrier |
| Diisopropyl sebacate (DUB | 5 | Carrier |
| DIS) | | |
| Active ingredients | 6 | Retinoid compounds, alpha-hydroxy |
| | | acids, beta-hydroxy acids, antiseptic |
| | | agents, antibiotic agents, anti- |
| | | inflammatory agents, antiviral agents, |
| | | antifungal agents, anti-acne agents, |
| | | sunscreen agents, plant extracts, |
| | | vitamins, corticosteriods, local |
| | | anesthetics, other bioactive |
| | | ingredients, ingredients that improve |
| | | consumer perception, pigments, etc. . . |

The composition may be prepared using the process described in Example 1.

Additional active ingredients may be added as desired.

### Example 8: Skin Care Product Composition with Strong Emollient Properties

A product composition is prepared with the composition specified in Formula H in Table 8. This composition provides strong emollient properties, which may be advantageous in some products and methods of use.

TABLE 8
Formula H

| Component (TRADE NAME) | Amount (% w/w) | Primary Purpose |
|---|---|---|
| Trisiloxane | 30 | Carrier |
| Dimethicone | 36 | Carrier |
| SD alcohol 40B, 200 proof | 20 | Carrier |
| Diisopropyl sebacate (DUB DIS) | 8 | Carrier |
| Active ingredients | 6 | Retinoid compounds, alpha- |
| | | hydroxy acids, beta-hydroxy acids, |
| | | antiseptic agents, antibiotic agents, |
| | | anti-inflammatory agents, antiviral |
| | | agents, antifungal agents, anti-acne |
| | | agents, sunscreen agents, plant |
| | | extracts, vitamins, corticosteriods, |
| | | local anesthetics, other bioactive |
| | | ingredients, ingredients that |
| | | improve consumer perception, |
| | | pigments, etc. . . |

The composition may be prepared using the process described in Example 1. Additional active ingredients may be added as desired.

### Example 9: Optical Clarity of Compositions

The optical clarity of the carrier compositions and product compositions may be measured using an ultraviolet (UV)-visible spectrometer according to the following procedures. Details of the practice of UV-visible spectroscopic measurements may be found, for example, in D. Skoog, et al., Principles of Instrumental Analysis, 6th Ed., Thomson Brooks/Cole Publishing, Belmont, Calif., 2007.

The spectra were collected using a PerkinElmer Lambda 25 UV-visible spectrometer (Waltham, Mass., USA). The samples were introduced into a 1 cm quartz cuvette using a glass pipette. A wavelength scan was performed over the visible spectral range from 400 to 700 nm for visible spectra and from 190 nm to 700 for UV-visible spectra. The slit width was set at 1 nm, the scan speed was 480 nm/min, the data interval was 1 nm, and 1 cycle was collected with a 1 second cycle time. The measurement data were collected in absorbance units and reprocessed in % transmittance units after data collection.

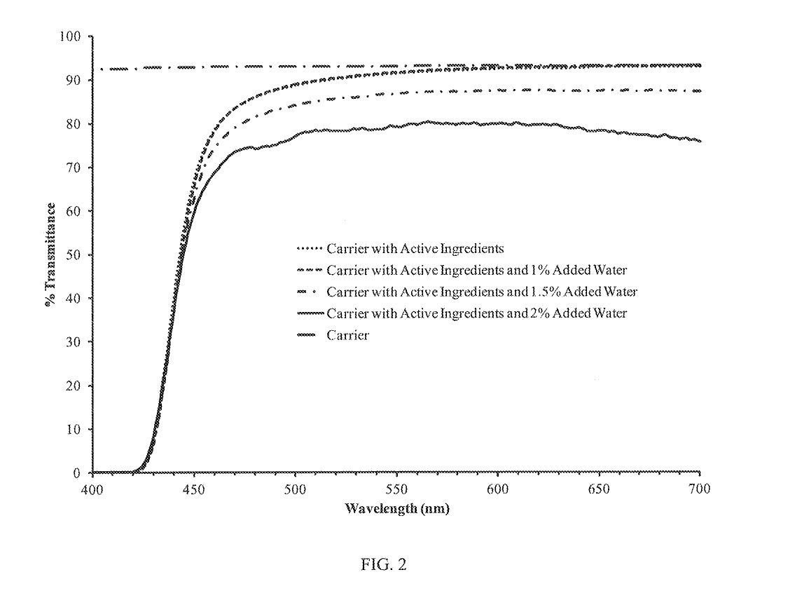

The results of the visible spectroscopic measurement on neat carrier and product compositions are shown in FIG. 2. The visible transmittance spectra are shown for a carrier composition (“Carrier”), a product composition prepared according to Formula E as given in Table 5 (“Carrier with Active Ingredients”), and product compositions prepared according to Formula E as given in Table 5 but with the addition of 1% (“Carrier with Active Ingredients with 1% Added Water”), 1.5% (“Carrier with Active Ingredients with 1.5% Added Water”), and 2% water (“Carrier with Active Ingredients with 2% Added Water”). The product compositions are observed to transmit light over nearly the entire visible region.

A UV-visible spectroscopic measurement was performed on a product composition prepared according to Formula E as given in Table 5 and, prior to UV-visible spectroscopic analysis, diluted 1:100 vol/vol in specially-denatured alcohol (“Carrier with Active Ingredients, 1:100 Dilution in SD Alcohol”). The product composition exhibits an absorbance with maximum wavelength of approximately 360 nm.

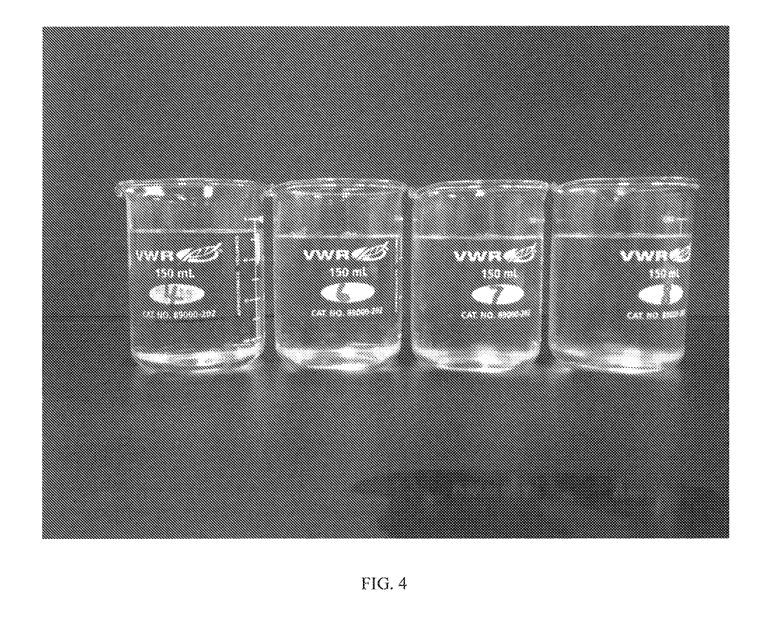

The optical appearance of solutions may be seen in the photograph of FIG. 4. Photographs of a composition prepared according to Formula E as given in Table 5 (solution labeled 4), and compositions prepared according to Formula E as given in Table 5 but with the addition of 1% water (solution labeled 6), 1.5% water (solution labeled 7), and 2% water (solution labeled 8) are shown.

### Example 10: Solubility of Hydrophobic and Hydrophilic Active Ingredients in Carriers

The solubility of several active ingredients, representing hydrophobic and hydrophilic compounds, was measured as shown in Table 9. The results demonstrate the unexpectedly superior solubilization capabilities of embodiments of the carriers.

TABLE 9
Solubility of Selected Active Ingredients in Carriers (NS = not soluble, S = soluble)

| | | | | SD40 | SD40 | SD40 |
|---|---|---|---|---|---|---|
| | | | | alcohol:PMX- | alcohol:PMX- | alcohol:PMX- |
| | | | | 1184¹:diisopropyl | 1184¹:Diisopropyl | 1184¹:diisopropyl |
| Active | Diisopropyl | PMX- | SD40 | sebacate | sebacate | sebacate |
| Ingredient | sebacate | 1184¹ | alcohol | (25:64:5)² | (10:79:5)² | (10:54:30)² |
| Azelaic acid, up | NS | NS | S | S | NS | NS |
| to 0.3% w/w | | | | | | |
| Mandelic acid, | NS | NS | S | S | NS | NS |
| up to 0.7% w/w | | | | | | |
| Niacinamide, | NS | NS | S | S | NS | NS |
| up to 0.2% w/w | | | | | | |
| Alfalfa | NS | NS | S | S | NS | NS |
| (medicago | | | | | | |
| sativa), chicory | | | | | | |
| (cichorium | | | | | | |
| intybus), water, | | | | | | |
| extract, up to | | | | | | |
| 1.5% w/w | | | | | | |
| Bakuchiol, up | S | S | S | S | S | S |
| to 1.5% w/w | | | | | | |
| 3-0-ethyl | NS | NS | S | S | NS | NS |
| ascorbic acid, | | | | | | |
| up to 2% w/w | | | | | | |
| ASYNTRA SL | S | S | S | S | S | S |
| (caprylic/capric | | | | | | |
| triglyceride, | | | | | | |
| hexyl | | | | | | |
| rescorcinol, | | | | | | |
| ethyl linoleate), | | | | | | |
| up to 1.5% w/w | | | | | | |

¹Silicone oil, XIAMETER PMX-1184 (Dow Corning).
²All values are given as ratios in units of % by weight, and additionally include 6% by weight of active ingredients.

### Example 11: An Eight-Week Randomized Evaluation Comparing the Effects of Four Test Products on Skin Condition

A clinical study was conducted to compare the composition of Table 3 (Formula C) to two competitor products and an aqueous skin care composition also prepared for the study as described in Table 4 (Formula D). The objective of this eight-week study randomized study was to evaluate and compare the ability of the four aforementioned skin care compositions to reduce features associated with aging in facial skin. A total of 64 subjects were enrolled in the study. Assessments were made based on visual attribute grading by a trained expert, instrumental analyses, imaging analysis, and participant questionnaires.

### Study Objectives

The objective of this investigation was to assess the ability of four anti-aging skin treatment products to reduce the signs of aging after one, four, and eight weeks of use as directed. Specific attention was paid to the following observations: (1) Firms facial skin after one, four, and eight weeks of treatment, as assessed by visual grading, CUTOMETER and Subjective questionnaire; (2) Reduces the appearance of fine lines/wrinkles in the crow's feet and peri-oral area after one, four, and eight weeks of treatment, as assessed by visual grading, subjective questionnaire and CLARITY Pro analysis (subgroup of 10 per product group); (3) Reduces facial skin discolorations (age spots) after one, four, and eight weeks of treatment, as assessed by visual grading, Subjective questionnaire and CLARITY Pro analysis; (4) Reduces facial skin redness after one, four, and eight weeks, as assessed by visual grading, Subjective questionnaire and CLARITY Pro analysis; (5) Increases facial skin radiance/luminosity after one, four, and eight weeks, as assessed by visual grading, subjective questionnaire and CLARITY Pro analysis; (6) Improves facial skin tone after one, four, and eight weeks, as assessed by visual grading and subjective questionnaire; (7) Improves facial skin texture/smoothness after one, four, and eight weeks, as assessed by visual grading, CUTOMETER and subjective questionnaire; (8) Minimizes the appearance of facial pore size after one, four, and eight weeks, as assessed by visual grading, subjective questionnaire and CLARITY Pro analysis; (9) Improves facial complexion health after one, four, and eight weeks, as assessed by visual grading, subjective questionnaire and CLARITY Pro analysis; (10) Improves overall facial skin appearance after one, four, and eight weeks, as assessed by visual grading, subjective questionnaire and CLARITY Pro analysis; (11) Improves facial skin hydration after one, four, and eight weeks, as assessed by visual grading, Moisture Meter instrumental evaluation and subjective questionnaire; (12) Improves facial skin elasticity/firmness after one, four, and eight weeks, as assessed by instrumental evaluation, CUTOMETER and subjective questionnaire; (13) Test product is X % as effective as the leading retinol/retinoid treatments in improving overall complexion health, as assessed by visual grading, subjective questionnaire and CLARITY Pro analysis (where “X %” is the quantity to be assessed by the clinical study); (14) Test product is well tolerated compared to the leading retinol/retinoid treatments without all the harshness and irritation associated with the leading retinol/retinoid treatments, as assessed by subjective tolerance, objective tolerance and subjective questionnaire; (15) Test product is X % as effective as the leading retinol/retinoid treatments in reducing lines/wrinkles, pore size and skin discolorations (age spots) and improving skin tone evenness, complexion health, texture/smoothness, radiance/luminosity, elasticity/firmness and redness as assessed by visual grading, subjective questionnaire and CLARITY Pro analysis; (16) X % of subjects showed an improvement in lines/wrinkles, skin discolorations (age spots), skin tone evenness, complexion health, texture/smoothness, radiance/luminosity, pore size, elasticity/firmness and redness after one, four, and eight weeks, as assessed by visual grading, CUTOMETER, subjective questionnaire and CLARITY Pro analysis

### Study Design

This study was an eight-week, randomized evaluation completing with no less than sixty subjects divided into four groups of at least fifteen subjects per product. All subjects were supplied with a daily cleanser and moisturizer with sun protection factor (SPF) in addition to one test product. A soap-only (CAMAY soap) washout period of one-week preceded the eight-week study period. Facial skin condition was evaluated instrumentally using the CUTOMETER and Moisture Meter. Additionally, CLARITY Pro analysis was performed on a subset of ten subjects per product group, for a total of forty. Visual clinical grading of skin attributes using standard ordinal scales and visual analogue scales (VAS) was also performed by a trained study technician. Consumer perception of product efficacy was collected utilizing subjective questionnaires. Evaluations occurred at week zero (baseline) and after one, four and eight weeks of test product use.

Participants in the study were divided into four study groups with 16 participants in each group. Each study group was provided with a different skin care composition or product. Group A was provided with the RETINOID product (Young Pharmaceuticals, Wethersfield, Conn., USA). Group B was provided with the commercial PHILOSOPHY MIRACLE WORKER product (Coty Inc., New York, N.Y., USA). Group C was provided with a composition prepared according to Formula C, as given in Table 3 of this application, and described in the foregoing Example 3 and elsewhere in this application. Group D was provided with a composition prepared according to Formula D, as given in Table 4 of this application, and described in the foregoing Example 4 and elsewhere in this application.

A sufficient number subjects were recruited to ensure completion with no less than sixty subjects or four panels of fifteen subjects per panel. At least 90% of subjects were Caucasian or light Hispanic and approximately 10% of subjects in each group was Asian. All subjects selected for participation in the study met the following inclusion and exclusion criteria for enrollment.

Subjects enrolled in the study met the following inclusion criteria: (1) healthy Caucasian females thirty to fifty-nine years of age (inclusive) at study enrollment; (2) self-reported Fitzpatrick Skin type I-IV; (3) able to understand and willing to: complete a brief personal medical history, review and sign an informed consent document and follow all study instructions; (4) fine lines/wrinkles in the crows' feet area OR peri-oral area, scoring ≧3.0 or ≦7.0 cm on a standard 10 cm visual analog scale (VAS) as well as an elasticity/firmness or tone score of ≧3.0 or ≦7.0 on a standard 10 cm VAS; (5) free of any dermatologic disorders that may have interfered with evaluation of test product performance; (6) willing to sign a model release document allowing study results including their images to be used for multi-media advertising purposes; (7) willing to use a supplied cleanser and moisturizer with SPF products and abstain from extended periods of facial sun exposure and all use of artificial tanning for the duration of the study; (8) willing to continue use of all regularly-used brands of color cosmetics provided they did not make any claims regarding moisturizing/anti-aging, and to refrain from beginning the use of any new facial products other than the assigned test materials; (9) willing to come to the lab without wearing makeup; (10) willing to not use any skin firming, anti-aging, anti-wrinkle, moisturizing skin lightening, or any other unassigned product or topical or systemic medication known to affect skin aging or dyschromia (products containing alpha/beta/poly-hydroxy acids, vitamin C, soy, Q-10, hydroquinone; systemic or topical retinoids, etc. . . . ) during the study other than the assigned test product; and (11) willing to refrain from any facial spa treatments including facials, microdermabrasion, chemical peels, BOTOX, etc. . . . during the study.

Subjects were excluded from the study if they met any of the following criteria: (1) Pregnant, breast-feeding, or planning a pregnancy during the study; (2) Regularly using (>3 times per week) antihistamine and/or anti-inflammatory medications; (3) Any history of sensitivity to skin treatment products; (4) Any condition(s) apparent at entry or recognized after entry that are likely to invalidate a subject's consent to participate in this study and/or limit the ability of a subject to regularly attend all study visits or to comply with all other protocol requirements such as: diseases, injuries, alcoholism, drug abuse, psychosis, antagonistic personality, poor motivation, infirmity disability, other problems that may be emotional, intellectual, psychological or social; (5) Any other condition(s) considered by the investigator as sound reasons for disqualification from enrollment into the study; and (6) an employee of the firm performing the study or other testing firms/laboratories, cosmetic or raw goods manufacturers or suppliers.

### Evaluation and Study Procedures

The screening and washout procedure was as follows. Subjects completed a brief medical/personal history, and read and signed an informed consent document prior to receiving any study instructions. For washout, all subjects were instructed to wash facial skin at least two times per day with CAMAY soap, using warm water to rinse. Subjects were also instructed to discontinue use of all other topical facial products except for non-moisturizing/anti-aging color cosmetics for the entire washout period. All subjects were instructed to wash their face at least one to two hours prior to each subsequent visit.

The week zero/baseline visit procedure was as follows. After arrival and visit check-in procedures, subjects sat at room temperature and humidity for at least fifteen minutes in order for their facial skin to equilibrate to indoor ambient conditions. Subjects will then undergo visual and instrumental baseline assessments. Subjects were considered qualified and were enrolled upon meeting all inclusion/exclusion criteria. All findings were documented on the appropriate case report form. Upon completion of the baseline evaluations, each subject received one of four study products per a prepared randomization scheme, a daily cleanser for use throughout the remainder of the study and a moisturizer with SPF, a diary and instructions for product use over the following twelve weeks. Subjects were advised to inform study center staff immediately if any reaction was noted on facial skin. Subjects were given an appointment time for the next three visits (weeks one, four and eight) and instructed to wash their face one to two hours prior. Subjects were then dismissed from the baseline visit.

The procedure followed at weeks one, four, and eight was as follows. After arrival and visit check-in procedures, subjects sat at room temperature and humidity for at least fifteen minutes in order for their facial skin to equilibrate to indoor ambient conditions. During this time they completed the subjective questionnaire. All subjects then underwent scheduled visual and instrumental assessments. Subjects were interviewed and/or subject diaries and test product were reviewed to ensure that any adverse experiences which may have occurred were recorded appropriately, product is being used correctly, and that a sufficient quantity of product remained for the duration of the study (except at week eight). All findings were documented on the appropriate case report form. At the week eight visit, subjects received a stipend for their participation after all visit requirements were met, including product and diary return. They were then dismissed from the study.

Visual evaluations were performed using the following procedure. Subjects' facial skin was clinically graded by a trained evaluator utilizing standard ordinal scales and visual analog scales, or VAS, at week zero (baseline), and at weeks one, four and eight. VAS is a method by which a numerical assessment value can be captured for subjective characteristics or attitudes that cannot be directly measured. When responding to a VAS item, respondents specify their level of agreement to a statement by indicating a position along a line (10 cm) between two end-points or anchor responses. Signs of photo aging can be categorized as follows: Mild=1-3.9, Moderate=4-6.9, Severe=7-10.

VAS for fine lines/wrinkles in the crow's feet and peri-oral area, tone, texture/smoothness, radiance/luminosity, elasticity/firmness and overall appearance were utilized. Except where noted, all visual assessments were global (both cheeks, forehead, nasolabial area, chin). The same trained visual evaluator performed all visual assessments throughout the study. The VAS to be utilized in this study was as follows. Fine lines/wrinkles (Global—crow's feet) were graded from none to numerous. Fine lines/wrinkles (Global—peri-oral area) were graded from none to numerous. Tone was graded from “even, healthy skin color” to “uneven, discolored appearance.” Texture/smoothness was graded from “smooth, silky” to “coarse, rough.” Radiance/luminosity was graded from “radiant, luminous appearance” to dull/matte and/or sallow appearance.” Elasticity/firmness (face/left cheek) was graded from “firm, tight-appearing with good stretch properties” to “loose-appearing with poor stretch properties.” Overall appearance was graded from “healthy looking” to “aged/unhealthy looking”

Ordinal scales assign a number to the quality of a given attribute. The following ordinal scales were utilized to assess dark spots I discolorations and product tolerance (dryness, erythema and edema) for this study. Subjective tolerance was also evaluated at all visits using ordinal scales. Dark spots/discolorations were graded as follows: 0.0, no color difference present, weak intensity (light brown); 1.0, mild, faint color; 2.0, moderate color difference and intensity (light brown/tan color); 3.0, moderate/deep color difference (brown/tan); 4.0, intense/deep color difference (dark brown/tan). Dryness was graded as follows: 0.0, normal skin, no signs of dryness; 1.0, mild, slight but definite dryness, fine scales present, may have powdery or ash appearance; 2.0, moderate, somewhat scaly, some cracking evident as uplifting scales; 3.0, marked, Marked coarse scaling, cracking evident as uplifting scales; 4.0, severe, Very coarse scaling, cracking progressing to fissuring, erythema may be present (test site discontinued from additional product applications). Erythema was graded as follows: 0, no erythema; 1, very slight erythema (barely perceptible); 2, well-defined erythema; 3, moderate to severe erythema; 4, severe erythema (beet redness) to slight eschar formation (injuries in depth, test site discontinued from additional product applications). Edema was graded as follows: 0, no edema; 1, very slight edema (barely perceptible); 2, slight edema (edges of area well-defined by definite raising); 3, moderate edema (raised approximately 1 mm); 4, severe edema (raised more than 1 mm and extending beyond area of exposure, test site discontinued from additional product applications). Symptoms of irritation were evaluated and reported by subjects for stinging. tingling, itching and burning utilizing the following scales: 0=none, 1=mild, 2=moderate, 3=severe.

The CUTOMETER, Moisture Meter and CLARITY Pro (subgroup often subjects per product group) were utilized to evaluate subjects' facial skin in this study. Subjects remained at indoor temperature and humidity for at least fifteen minutes prior to any instrumental assessment in order to ensure equilibration of their skin indoor ambient conditions.

The CUTOMETER (Firmness/Elasticity) instrument applies a negative pressure to the skin surface and calculates the height to which the skin can be drawn up and the rate at which it returns to equilibrium thus providing a measurement of firmness and elasticity through wave form analysis. The standard measurements of waveform variables R0 for firmness and R5 for elasticity were used. Firmness and elasticity were measured on the right side of the face on all subjects at all visits.

The measurement of facial skin surface hydration at stratum corneum level will be assessed using the MoistureMeterSC, a laboratory-grade instrument that measures skin hydration with precision and accuracy. The MoistureMeterSC measures the capacitance of the layered structure composed of the probe, stratum corneum and the underlying skin layers. The measured capacitance is directly proportional to the water content of stratum corneum. The measurement frequency is 1.25 MHz. The MoistureMeterSC's effective measurement depth depends on the thickness of the dry layer and is thus individual. MoistureMeter measurements were performed on the left side of the face for all subjects at all visits.

The CLARITY Pro Study Manager imaging system captures full face, frontal and 45° lateral, images in multi-spectral lighting. White light and deep blue light images reveal skin conditions on and beneath the skin's surface layer. The system uses skin feature recognition and extraction to allow for subsequent skin analysis. One frontal and one left lateral image was taken at each visit for each subject in a subgroup of ten from each product group, forty subjects total. Resulting images were analyzed for wrinkles (left): total wrinkle count, fine line count, deep line count, crows' feet and peri-oral area wrinkle length, width and severity; Pigmentation (left): total spots, area affected, pigment evenness; Complexion (front): complexion health, radiance; and Redness (front): subsurface erythema Pores (Left): pore count, pore size, pore visibility. Regarding CLARITY Pro analysis for wrinkle length and width: the software for CLARITY captures and evaluates wrinkle width and length based on pixels. The calculations of length and width are provided for the specific time point of measurement. Therefore, in order to compare to baseline, a novel mathematical algorithm will be used to approximate changes in average severity, length and width between time points. For CLARITY Pro assessments, photos of a total of five subjects from the sub-group with the greatest clinical improvement in wrinkle length, width, and severity (indicated by assessment results) will be provided to sponsor. These photos will analyze fine lines/wrinkles (count width, length and severity), pore size, count and visibility and complexion health/radiance.

A subjective questionnaire was administered to all subjects at the week one, week four and week eight visits.

### Study Results

The study found no significant different in subject tolerance for any of the four groups. Tolerance was assessed as an increase in dryness, erythema, edema, stinging, tingling, itching, or burning. No statistically significant worsening over time was found for any test group. No statistical difference between any of the groups was noted with respect to tolerance.

The overall results of the visual skin attribute grading are summarized as follows, where each group showing a significant improvement is discussed and all other groups not discussed did not show a significant improvement. Visual skin attribute grading was performed to assess dark spots, lines and wrinkles (including crow's feet and peri-oral lines and wrinkles), tone, texture, radiance, and overall appearance. At week 4, a significant improvement in overall appearance was observed for Groups B and D. At week 8, dark spots, lines and wrinkles (including crow's feet and peri-oral lines and wrinkles), tone, texture, radiance, and overall appearance were significantly improved for Group A. Also at week 8, lines and wrinkles (including crow's feet and peri-oral lines and wrinkles), tone, texture, radiance, and overall appearance were significantly improved for Group B. In addition, at week 8, lines and wrinkles (including crow's feet and peri-oral lines and wrinkles) and texture were improved for Group D.

The overall results of the instrumental analyses are summarized as follows, where each group showing a significant improvement is discussed and all other groups not discussed did not show a significant improvement. Significant improvement in skin hydration was observed at week 1 for Groups A and B. Significant improvement in skin hydration at week 8 was observed for Group D. Significant improvement in skin firmness was observed at week 8 for Group C.

The overall results of the imaging analyses using the CLARITY Pro system are summarized as follows, where each group showing a significant improvement is discussed and all other groups not discussed did not show a significant improvement. At week 1, Group A showed improvement in the average length of lines and wrinkles, Group B showed an improvement in average pore visibility, complexion health, skin radiance and luminosity, and overall redness, Group C showed an improvement in total lines and wrinkles and fine lines, pore count, average pore size, average pore visibility, complexion health, skin radiance and luminosity, and redness variation. At week 4, Group A showed an improvement in pore count and average pore size, Group B showed an improvement in pore count and skin radiance and luminosity, Group C showed an improvement in total lines and wrinkles and fine lines, pore count, average pore size, average pore visibility, complexion health, skin radiance and luminosity, and redness variation, and Group D showed an improvement in skin radiance and luminosity. In all instances at 4 weeks, products C and D scores showed greater improvement than product A. At week 8, Group A showed an improvement in wrinkle severity and skin radiance and luminosity, Group B showed an improvement in wrinkle severity, pore count, and skin radiance and luminosity, Group C showed an improvement in average length and width of wrinkles, number of fine lines, pore count, complexion health, skin radiance and luminosity, overall redness, and redness variation.

The detailed study results from the CLARITY Pro system are reported in Tables 9, 10 and 11.

The subject questionnaire results indicated no significant difference between the four groups.

### Example 12: Consumer Perception and Clinical Efficacy of a Skin Treatment Product

A clinical study was conducted using the compositions described in Formula E, Table 5, and Example 5. The study was conducted using 50 female subjects to determine if the composition, when used once daily for 8 weeks, improved the depth and width (area) of fine lines and wrinkles, skin clarity, skin blotchiness, skin hydration, skin firmness, and skin elasticity. The objectives of this study were to determine if the use of a skin treatment product: (1) reduced the depth (area) of fine lines/wrinkles; (2) reduced the width (area) of fine lines/wrinkles; (3) improved skin clarity/blotchiness; (4) increased skin hydration; and (5) improved skin firmness/elasticity. The 50 female subjects were aged 33-55 years (median age 38-42). The evaluations were performed after 2, 4 and 8 weeks of product use, except that skin hydration was additionally evaluated immediately after a single application of the test product and 30 minutes, 1, 2 and 3 hours post-application.

### Study Design and Evaluation Procedures

The following inclusion criteria were applied to select subjects for the study: (1) Females between the ages of 33 and 55 (inclusive) in general good health (no physical required); (2) Individuals with a global fine line score of “5” (noticeable) or greater on the face (for qualification purposes only); (3) Individuals with a global wrinkle score of “5” (noticeable) or greater on the face (for qualification purposed only); (4) Individuals with a skin clarity score of −5″ (moderate areas of discoloration visible) or greater on the face (for qualification purposes only); (5) Individuals with a skin blotchiness score of “5” or greater (moderate areas of blotchiness/uneven skin tone) on the face; (6) Individuals who could read, understand and sign the Informed Consent; and (7) Individuals with anticipated ability to follow the study directions, to participate in the study, to return for all visits and to apply the product as per instructions.

The following exclusion criteria were applied to select subjects for the study: (1) Women who were pregnant, planning a pregnancy, lactating and/or nursing a child; (2) Individuals with any visible skin disease; (3) Individuals with sunburn, suntan on the face or planning a vacation with sun-exposure or planning the use of a tanning booth during the course of the study; (4) Individuals engaged in a concurrent research project of a facial product; (5) Individuals taking medications which might have interfered with the test results including the use of steroidal/non-steroidal anti-inflammatory drugs or antihistamines; (6) Individuals who had undergone a laser resurfacing or dermabrasion procedure on the face in the past 2 years or a chemical face peel (deep peel in the past 1 year; superficial peel in the past 2 months); (7) Individuals with acne, active atopic dermatitis/eczema or psoriasis; (8) Individuals who had a surgical “cosmetic” procedure on the face within the past 10 years; (9) Treatment or history of any type of cancer. (10) Individuals who were under treatment for asthma or diabetes; (11) Individuals with a known sensitivity to cosmetics or personal care products.

The study was designed as an 8-week study, in which the test product was used by each of the test subjects according to the Sponsor's use instructions. Subjects reported to the Testing Facility for the baseline visit. A trained technician globally evaluated lines, wrinkles, skin clarity and blotchiness on the face of each subject to determine qualification. A CUTOMETER (Courage+Khazaka, Germany) measurement was taken on the face of each subject to measure skin elasticity/firmness and a CORNEOMETER (Courage+Khazaka) measurement was taken on the face to measure skin hydration. Digital photographs were taken of the face of each subject and the photographs were analyzed to determine changes (if any) in the appearance of the depth and width (area) of fine lines/wrinkles and skin clarity/blotchiness. An irritation evaluation was also conducted for safety purposes. Subjects were given the test product, use instructions and a daily diary and they were instructed to report to the Testing Facility after 2, 4 and 8 weeks of product use for additional evaluations visual evaluations, photographs and instrumental measurements. Subjects were required to complete a questionnaire after 2, 4 and 8 weeks of product use.

Additionally, at the baseline visit only, the test product was applied under the supervision of a trained technician. Immediately after application and 30 minutes, 1, 2 and 3 hours post-application, CORNEOMETER readings were taken to measure skin hydration.

Evaluations of efficacy were based on a comparison of a baseline evaluation versus each observation period. Baseline evaluations were performed as follows. Subjects reported to the Testing Facility for the baseline visit with a freshly washed “clean” face (without wearing cosmetics or having applied any skin care products) for baseline visual assessments, photographs, and instrumental measurements. Evaluations were conducted according to the procedures described below.

Subjects were given the test product to take home and a daily diary with the following instructions: The following must be included in this diary: 1. The dates and times (p.m.) the product was used. 2. Any comments or observations you may have had while using the regimen. 3. DO NOT USE ANY NEW SKIN CARE PRODUCTS OR COSMETICS DURING THE TEST PERIOD. 4. DO NOT USE ANY OTHER RETINOL PADS ON YOUR FACE DURING THE ENTIRE TEST PROCEDURE. 5. Apply the product according to the directions below: “Use once daily in the evenings. Pour solution slowly over pads. Wipe pad over face in gentle upward and outward motions. Apply sunscreen daily in the morning. Caution: For external use only. Use only as directed. Keep away from eyes and mouth. If contact occurs, rinse well with water. If irritation, redness or itching occurs, contact the Testing Facility immediately. Do not use if pregnant or nursing. Keep out of reach of children.”

Two-, four- and eight-week evaluations were performed as follows. Follow-up visual evaluations, instrumental measurements and digital photographs were taken after 2, 4 and 8 weeks of product use. Additionally, subjects completed a questionnaire at the 2-, 4- and α-week visits.

Global evaluation of fine lines was performed as follows. For qualification purposes only, a trained technician globally evaluated fine lines on the face of each subject according to the Scale for Scoring Fine Lines: 0=None; 1-3=Slight; 4-6=Noticeable; 7-9=Very Noticeable.

Global evaluation of wrinkles was performed as follows. For qualification purposes only, a trained technician globally evaluated wrinkles on the face of each subject according to the Scale for Scoring Wrinkles: 0=None; 1-3=Slight; 4-6=Noticeable; 7-9=Very Noticeable.

Global evaluation of skin blotchiness was performed as follows. For qualification purposes only, a trained technician evaluated skin blotchiness on the face of each subject according to the Scale for Scoring Skin Blotchiness: 0=Perfectly even skin tone (no discolorations visible); 1-3=Slight areas of blotchiness visible; 4-6=Moderate areas of blotchiness visible; 7-9=Severe areas of blotchiness visible.

Evaluation of skin clarity was performed as follows. For qualification purposes only, a trained technician evaluated skin clarity on the face of each subject according to the Scale for Scoring Skin Clarity: 0=Perfectly even complexion (no discolorations visible); 1-3=Slight areas of discoloration visible; 4-6=Moderate areas of discoloration visible; 7-9=Severe areas of discoloration visible.

CUTOMETER measurements were performed according to the following procedure. At baseline and after 2, 4 and 8 weeks of product use, the elasticity/firmness of the skin was measured on the face of each subject using the CUTOMETER. An increase in CUTOMETER measurements indicated an improvement (increase) in skin elasticity/firmness. A decrease represented a worsening.

CORNEOMETER measurements were performed according to the following procedure. At baseline (prior to product application) and 30 minutes, 1, 2 and 3 hours after single application and after 2, 4 and 8 weeks of product use, the moisture content of the skin was measured on the face of each subject using the CORNEOMETER. An increase in CORNEOMETER measurements indicated an improvement (increase) in skin moisture. A decrease represented a worsening.

Evaluation of irritation was performed at each evaluation, wherein a trained technician evaluated the face of each subject for irritation according to the scale below. This evaluation was for safety purposes only and was not used in determining efficacy. Scale for Scoring Irritation: 0=No irritation present; +=Barely perceptible irritation present; 1=Mild irritation present; 2=Moderate irritation present; 3=Marked irritation present; 4=Severe irritation present.

Digital photography procedure and analysis was performed according to the following procedure. At each visit, digital images of the face of each subject were taken from the front, right and left views using the VISIA CR 2.2 (Canfield Scientific, Fairfield, N.J.). In order to ensure consistency between the photographs, each subject was draped with a black cloth around the shoulders in order to eliminate the appearance of clothing in the pictures and each subject wore a black headband to pull hair off of and away from the face. The images were analyzed using IMAGE PRO software (MediaCybernetics, Bethesda, Md.) to determine changes (if any) in the following parameters: (1) depth and width (area) of fine lines; (2) depth and width (area) of wrinkles; and (3) skin clarity/blotchiness. In order to determine changes in the depth and width (area) of fine lines, the depth and width of the fine lines were measured (in pixels). A decrease in the mean pixel count represented an improvement. An increase represented a worsening. In order to determine changes in the depth and width (area) of wrinkles, the depth and width of the fine lines were measured (in pixels). A decrease in the mean pixel count represented an improvement. An increase represented a worsening. In order to determine changes in skin clarity/blotchiness, chroma was analyzed. The degree to which a color is free from being mixed with other colors is a good indication of its chromacity. An increase in the chroma score represented an improvement in skin clarity/blotchiness. A decrease represented a worsening.

Subject questionnaires were also included as part of the study. At the 2-, 4- and 8-week visits, subjects were required to respond to a questionnaire.

### Study Results

Fifty-one (51) female subjects between the ages of 34 and 56 years were empanelled. A total of 50 (50/51) subjects successfully completed the study. One test panelist (Subject No. 23) was discontinued for personal reasons unrelated to the conduct of the study. A protocol deviation occurred when one (1/51) test panelist (Subject No. 18) was enrolled outside the age range specified in the inclusion criteria. (Subject No. 18 was 56 years of age.) This deviation did not affect the conduct of the study.

At each visit, a trained technician measured skin firmness on the face of each subject using the CUTOMETER. The CUTOMETER evaluation results are shown in Table 12.

TABLE 12
Mean CUTOMETER Measurements
Mean Score ± Standard Deviation (S.D.), Mean % Change from Baseline
and % of Subjects with improvement from Baseline

| | | | | % of subjects |
|---|---|---|---|---|
| | | | | with |
| | Mean Score = | | Mean % Change | Improvement |
| | S.D. | p-value | from baseline | from baseline |
| Baseline | 0.511 ± 0.599 | — | — | — |
| Week 2 | 0.584* ± 0.048 | <0.001 | 15.3% | 100% |
| Week 4 | 0.664* ± 0.052 | <0.001 | 31.4% | 100% |
| Week 8 | 0.708* ± 0.045 | <0.001 | 40.1% | 100% |

*Statistically significant change from baseline p ≦ 0.05

When measurements taken after 2, 4 and 8 weeks of product use were compared with baseline measurements, there were highly significant (p<0.001) mean percent improvements of 13.3%, 31.4% and 40.1%, respectively, in skin elasticity/firmness based on CUTOMETER measurements. A total of 100% of the subjects showed improvement after 2, 4 and a weeks of product use.

At each visit, a trained technician measured skin moisture on the face of each subject using the CORNEOMETER. The CORNEOMETER evaluation results are shown in Table 13.

TABLE 13
Mean CORNEOMETER Measurements
Mean Score ± S.D., Mean % Change from Baseline
and % of Subjects with Improvement from Baseline

| | | | | % of subjects |
|---|---|---|---|---|
| | Mean | | | with |
| | Score = | | Mean % Change | Improvement |
| | S.D. | p-value | from baseline | from baseline |
| Baseline | 20.2 ± 7.3 | — | — | — |
| 30 Mins. | 30.6* ± 9.3 | <0.001 | 64.0% | 98.0% |
| 1 Hour | 40.3* ± 11.4 | <0.001 | 121.3% | 100% |
| 2 Hours | 44.3* ± 13.0 | <0.001 | 145.5% | 96.0% |
| 3 Hours | 47.9* ± 13.4 | <0.001 | 167.7% | 98.0% |
| Week 2 | 47.4* ± 10.1 | <0.001 | 164.1% | 100% |
| Week 4 | 48.5* ± 9.7 | <0.001 | 172.8% | 100% |
| Week 8 | 51.4* ± 9.5 | <0.001 | 191.9% | 100% |

*Statistically significant change from baseline p ≦ 0.05

When measurements taken after 30 minutes, 1, 2 and 3 hours following a single application and after 2, 4 and 8 weeks of product use were compared with baseline measurements, there were highly significant (p<0.001) mean percent improvements of 64.0%, 121.3%, 145.5%, 167.7%, 164.1%, 172.8% and 191.9% respectively, in skin moisture based on CORNEOMETER measurements. A total of 98.0% of the subjects showed improvement at 30 minutes and 3 hours, 96.0% showed improvement at 2 hours and 100% of subjects showed improvement after 1 hour and after 2, 4 and 8 weeks of product use.

At each visit, a trained technician evaluated irritation on the face of each subject. Table 14 presents a summary of mean irritation scores.

TABLE 14
Mean Irritation Scores
Mean Score ± S.D., Mean % Change from Baseline

| | Mean | | Mean % Change from |
|---|---|---|---|
| | Score ± S.D. | p-value | baseline |
| Baseline | 0.0 ± 0.0 | — | — |
| Week 2 | 0.0 ± 0.0 | 1.000 | 0% |
| Week 4 | 0.0 ± 0.0 | 1.000 | 0% |
| Week 8 | 0.0 ± 0.0 | 1.000 | 0% |

There was no irritation observed on any subject during the course of the study.

After 2, 4 and 8 weeks of product use, a trained technician took digital images on the face of each subject. Using IMAGE PRO software, the images were analyzed to determine changes in the area of fine lines. Table 15 presents a summary of the mean fine line image analysis.

TABLE 15
Mean Fine Line Area Image Analysis Scores
Mean Score ± S.D., Mean % Change from Baseline
and % Change with Improvement from Baseline

| | | | | % of Subjects |
|---|---|---|---|---|
| | Mean | | | with |
| | Score ± | p- | Mean % Change | Improvement |
| | S.D. | value | from baseline | from baseline |
| Baseline | 525.8 ± 88.5 | — | — | — |
| Week 2 | 507.9 ± 122.1 | 0.135 | −3.0% | 58.8% |
| Week 4 | 467.4 ± 105.6 | <0.001 | −9.8% | 74.5% |
| Week 8 | 288.7 ± 34.4 | <0.001 | −43.7% | 100% |

*Statistically significant change from baseline p ≦ 0.05

When images taken after 2, 4 and 8 weeks of product use were compared with baseline images, there were highly significant (p<0.001) mean percent improvements of 9.8% and 43.7% after 4 and 8 weeks, respectively, in fine line area based on image analysis. A total of 58.8%, 74.5% and 100% of subjects showed improvement after 2, 4 and 8 weeks of product use, respectively.

After 2, 4 and 8 weeks of product use, a trained technician took digital images on the face of each subject. Using IMAGE PRO software, the images were analyzed to determine changes in the area of wrinkles Table 16 presents a summary of the mean wrinkle image analysis.

TABLE 16
Mean Wrinkle Area Image Analysis Scores
Mean Score ± S.D., Mean % Change from Baseline
and % of Subjects with Improvement from Baseline

| | | | | % of Subjects |
|---|---|---|---|---|
| | Mean | | | with |
| | Scores ± | | Mean % Change | Improvement |
| | S.D. | p-value | from Baseline | from Baseline |
| Baseline | 427.9 ± 64.2 | — | — | — |
| Week 2 | 395.1* ± 61.8 | <0.001 | −7.2% | 74.5% |
| Week 4 | 375.6* ± 50.5 | <0.001 | −11.2% | 90.2% |
| Week 8 | 296.8* ± 232.2 | <0.001 | −29.2% | 100% |

*Statistically significant change from baseline p ≦ 0.05

When images taken after 2, 4 and 8 weeks of product use were compared with baseline images, there were highly significant (p<0.001) mean percent improvements of 7.2%, 11.2% and 29.2% respectively, in wrinkle area based on image analysis. A total of 74.5%, 90.2% and 100% of subjects showed improvement after 2, 4 and 8 weeks of product use, respectively.

After 2, 4 and 8 weeks of product use, a trained technician took digital images on the face of each subject. Using IMAGE PRO software, the images were analyzed to determine changes in skin clarity/blotchiness. Table 17 presents a summary of the mean skin clarity/blotchiness analysis.

TABLE 17
Mean Skin Clarity/Blotchiness Image Analysis Scores
Mean Scores ± S.D., Mean % Change from Baseline
and % of Subjects with Improvement from Baseline

| | | | | % of Subjects |
|---|---|---|---|---|
| | Mean | | | with |
| | Scores ± | | Mean % Change | Improvement |
| | S.D. | p-value | from Baseline | from Baseline |
| Baseline | 25.2 ± 2.9 | — | — | — |
| Week 2 | 25.3 ± 2.8 | 0.148 | 0.9% | 56.9% |
| Week 4 | 25.5* ± 2.9 | 0.007 | 1.6% | 74.5% |
| Week 8 | 26.2* ± 2.7 | <0.001 | 4.2% | 80.0% |

*Statistically significant change from baseline p ≦ 0.05

When images taken after 2, 4 and 8 weeks of product use were compared with baseline images, there were statistically significant (p<0.05) mean percent improvements of 1.6% and 4.2% after 4 and 8 weeks, respectively, in skin clarity/blotchiness based on image analysis. A total of 56.9%, 74.5% and 80.0% of subjects showed improvement after 2, 4 and B weeks of product use, respectively.

The overall conclusions of the study are summarized as follows. Skin firmness and skin elasticity was improved after 2, 4, and 8 weeks of treatment with the composition based on instrumental analysis. Skin hydration was significantly improved after 30 minutes and 1, 2 and 3 hours following a single application of the composition and after 2, 4, and 8 weeks of treatment with the composition based on instrumental analysis. The area of fine lines was significantly improved after 4 and 8 weeks of treatment with the composition, based on image analysis. The area of wrinkles was significantly improved after 2, 4, and 8 weeks of treatment with the composition based on image analysis. Skin clarity and skin blotchiness was significantly improved after 4 and 8 weeks of treatment based on image analysis.

### Example 13: Clinical Irritation Testing for Skin Treatment Products

Clinical irritation testing studies were performed on Formula C, as described in Example 3 and Table 3, and Formula E, as described in Example 5 and Table 5. The objective of these studies was to determine the irritation and/or sensitization potential of the each test formula after repeated application under occlusive patch test conditions to the skin of human subjects (non-exclusive panel). Informed Consent was obtained from each subject in the study and documented in writing before participation in the study. A copy of the Informed Consent was provided to each subject. A target enrollment of at least 50 male and female subjects ranging in age from 18 to 79 years was set for the test of each of the two formulas. The subjects chosen were to be dependable and were able to read and understand instructions. The subjects were not to exhibit any physical or dermatologic condition that would have precluded application of the test article or determination of potential effects of each test formula.

### Procedure

The study made use of the “9 Repeated Insult (occlusive) Patch Test” (9-RIPT), which is described in Marzulil, F. N., Maibach, H. I. Contact allergy: predictive testing in man, Contact Dermatitis 2, 1-17 (1976). An induction phase and a challenge phase were employed.

The following procedure was used during the induction phase testing of Formula C. A sufficient amount of Formula C (approximately 0.2 mL) was placed onto a 2 cm by 2 cm square of Webril® cotton fabric (approximately 0.05 mL/cm² of test material) affixed to Scanpor (Allerderm) semi-occlusive surgical tape. The patch was then applied to the back of each subject between the scapulae and waist, adjacent to the spinal mid-line. This procedure was performed by a trained technician/examiner and repeated every Monday, Wednesday and Friday until 9 applications of the test article had been made.

The following procedure was used during the induction phase testing of Formula E. A sufficient amount of the test article (approximately 0.2 mL) was placed onto a Parke-Davis Readi-Bandage® occlusive patch (approximately 0.05 mL/cm² of test material), which was applied to the back of each subject between the scapulae and waist, adjacent to the spinal mid-line. This procedure was performed by a trained technician/examiner and repeated every Monday, Wednesday and Friday until 9 applications of the test article had been made.

During the induction phases for both Formula C and Formula E, the following procedures were employed. The subjects were instructed to remove the patch 24 hours after application. Twenty-four hour rest periods followed the Tuesday and Thursday removals and 48-hour rest periods followed each Saturday removal. Subjects returned to the Testing Facility and the site was scored by a trained examiner just prior to the next patch application. If a subject were to develop a positive reaction of a level 2 erythema or greater during the Induction phase or if, at the discretion of the Study Director, the skin response warranted a change in site, the patch would be applied to a previously unpatched, adjacent site for the next application. If a level 2 reaction or greater occurred at the new site, no further applications would be made. Any reactive subjects would be subsequently tested during the challenge phase.

During the challenge phases for both Formula C and Formula E, the following procedures were employed. After a rest period of approximately 2 weeks (no applications of the test article), the challenge patch was applied to a previously unpatched (virgin) test site. The site was scored 24 and 72 hours after application. All subjects were instructed to report any delayed skin reactivity that occurred after the final challenge patch reading. When warranted, selected test subjects were called back to the clinic for additional examinations and scoring to determine possible increases or decreases in challenge patch reactivity.

Dermal responses for both the induction and challenge phases of the study were scored according to the following 6-point scale: 0=No evidence of any effect, +=Barely perceptible (Minimal, faint, uniform or spotty erythema), 1=Mild (Pink, uniform erythema covering most of the contact site), 2=Moderate (Pink-red erythema uniform in the entire contact site), 3=Marked (Bright red erythema with/without petechiae or papules), 4=Severe (Deep red erythema with/without vesiculation or weeping). All other observed dermal sequelae (e.g., edema, dryness, hypo- or hyperpigmentation) were appropriately recorded on the data sheet and described as mild, moderate or severe.

Data interpretation was performed using the following procedures. Edema, vesicles, papules and/or erythema that persist or increase in intensity either during the induction and/or challenge phase may be indicative of allergic contact dermatitis. Allergic responses normally do not resolve or improve markedly at 72-96 hours. Exceptions to typical skin reactions may occur. These may include, but not are limited to, symptoms of allergic contact sensitivity early in the induction period to one or more test products. When this occurs in one subject, such a reaction usually suggests either an idiosyncratic response or that the subject had a pre-exposure and/or sensitization to the test material or component(s) of the test material or a cross-reactivity with a similar product and/or component. Data for such reactions will be included in the study report but will not be included in the final study analysis/conclusion of sensitization.

### Study Results for Formula C

A total of 55 subjects (15 males and 40 females ranging in age from 19 to 79 years) were empanelled for the test procedure. Fifty-three (53/55) subjects satisfactorily completed the test procedure on Formula C. Two (2/55) subjects discontinued for personal reasons unrelated to the conduct of the study. Discontinued subject data were evaluated up to the point of discontinuation, but are not used in the conclusions of the study.

There was no skin reactivity observed at any time during the course of the study. During both the induction and challenge phases, no subject received a dermal response score other than 0. There was no evidence of irritation during the induction phase, and no evidence of sensitization during the challenge phase. In conclusion, under the conditions of a repeated insult (occlusive) patch test procedure conducted in 54 subjects, Formula E was not associated with skin irritation or allergic contact dermatitis in human subjects.

There was no skin reactivity observed at any time during the course of the study. During both the induction and challenge phases, no subject received a dermal response score other than 0. There was no evidence of irritation during the induction phase, and no evidence of sensitization during the challenge phase. In conclusion, under the conditions of a repeated insult (semi-occlusive) patch test procedure conducted in 53 subjects, Formula C was not associated with skin irritation or allergic contact dermatitis in human subjects.

### Study Results for Formula E

A total of 55 subjects (16 males and 39 females ranging in age from 18 to 71 years) were empanelled for the test procedure. Fifty-four (54/55) subjects satisfactorily completed the test procedure on Formula E. One (1/55) subject discontinued for personal reasons unrelated to the conduct of the study. Discontinued subject data were evaluated up to the point of discontinuation, but are not used in the conclusions of the study.

There was no skin reactivity observed at any time during the course of the study. During both the induction and challenge phases, no subject received a dermal response score other than 0. There was no evidence of irritation during the induction phase, and no evidence of sensitization during the challenge phase. In conclusion, under the conditions of a repeated insult (occlusive) patch test procedure conducted in 54 subjects, Formula E was not associated with skin irritation or allergic contact dermatitis in human subjects.

All references disclosed herein are fully and completely incorporated by reference as if set forth in their entirety.

1. A product composition having a carrier composition comprising:
(a) a volatile silicone in a concentration of 40% by weight to 70% by weight of the product composition,
(b) an organic alcohol in a concentration of 10% by weight to 30% by weight of the product composition, and
(c) a compound according to Formula I 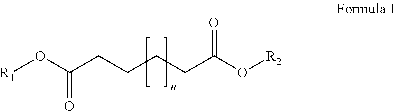
in a concentration of 1% by weight to 10% by weight of the product composition, wherein R₁ and R₂ are independently selected from the group consisting of isopropyl, propyl, and ethyl, and wherein n is from 1 to 6, and wherein the carrier composition is an optically transparent liquid.

2. The product composition of claim 1, wherein the carrier composition comprises at least 90% by weight of the product composition.

3. The product composition of claim 1, wherein the volatile silicone comprises:
(a) decamethyltetrasiloxane in a concentration of 32% by weight to 38% by weight of the volatile silicone,
(b) octamethyltrisiloxane in a concentration of 29% by weight to 35% by weight of the volatile silicone,
(c) dodecamethylpentasiloxane in a concentration of 12% by weight to 20% by weight of the volatile silicone, and
(d) polydimethylsiloxane in a concentration of 12% by weight to 20% by weight of the volatile silicone.

4. The product composition of claim 1, wherein the organic alcohol is selected from the group consisting of ethanol, specially-denatured alcohol, and isopropanol.

5. The product composition of claim 1, wherein the compound according to Formula I is selected from the group consisting of diisopropyl sebacate, diisopropyl nonanedioate, diisopropyl octanedioate, diisopropyl heptanedioate, diisopropyl adipate, and combinations thereof.

6. The product composition of claim 1, wherein the product composition comprises up to about 6% by weight of at least one active ingredient.

7. The product composition of claim 1, wherein the product composition further comprises an active ingredient selected from the group consisting of niacinamide, mandelic acid, azelaic acid, hydroxypinacolone retinoate, dimethyl isosorbide, bakuchiol, bisabolol zingiber officinale (ginger) root extract, or combinations thereof.

8. The product composition of claim 7, wherein the product composition comprises 0.05 to 0.5% by weight of niacinamide.

9. The product composition of claim 7, wherein the product composition comprises 0.05 to 0.5% by weight of azelaic acid.

10. The product composition of claim 7, wherein the product composition comprises 0.1 to 1.0% by weight of mandelic acid.

11. The product composition of claim 7, wherein the product composition comprises 0.5 to 1.2% by weight of bisabolol zingiber officinale (ginger) root extract.

12. The product composition of claim 7, wherein the product composition comprises 0.5 to 2.0% by weight of bakuchiol.

13. The product composition of claim 7, wherein the product composition comprises 0.7 to 1.1% by weight of a mixture, the mixture comprising 90% by weight of dimethyl isosorbide and 10% by weight of hydroxypinacolone retinoate.

14. The product composition of claim 1, further comprising a water soluble active ingredient and an oil soluble active ingredient.

15. The product composition of claim 14, wherein the water soluble active ingredient is selected from the group consisting of an alpha hydroxy acid, vitamin B, vitamin C, an antibiotic drug, an anti-infective compound, an aqueous plant extract, and combinations thereof, and wherein the oil soluble active ingredient is selected from the group consisting of a corticosteroid, vitamin A, vitamin D, vitamin E, an anti-inflammatory compound, a skin whitening ingredient, a sunscreen agent and combinations thereof.

16. The product composition of claim 1, wherein the product composition includes a carrier composition that has a transmittance selected from the group consisting of greater than 50% at 450 nm, greater than 75% at 500 nm, greater than 85% at 550 nm, greater than 90% at 600 nm, greater than 90% at 650 nm, greater than 90% at 700 nm, and combinations thereof.

17. A method of improving skin firmness in a human, the method comprising topically administering the product composition according to claim 1 to the skin of the human.

18. The method of claim 17, wherein the product composition is administered once a day for a period of 2, 4, or 8 weeks.

19. The method of claim 17, wherein the skin firmness is improved by at least 10%.

20. A method of improving skin hydration in a human, the method comprising topically administering the product composition according to claim 1 to the skin of the human.

21. The method of claim 20, wherein the product composition is administered once a day for a period of 2, 4, or 8 weeks.

22. The method of claim 20, wherein the skin hydration is improved by at least 50%.

23. A method of reducing mean fine line area in the skin of a human, the method comprising topically administering the product composition according to claim 1 to the skin of the human.

24. The method of claim 23, wherein the product composition is administered once a day for a period of 2, 4, or 8 weeks.

25. The method of claim 23, wherein the mean fine line area is improved by at least 8%.

26. A method of reducing mean area of skin wrinkles in a human, the method comprising topically administering the product composition according to claim 1 to the skin of the human.

27. The method of claim 26, wherein the product composition is administered once a day for a period of 2, 4, or 8 weeks.

28. The method of claim 26, wherein the mean area of skin wrinkles is improved by at least 6%.

29. A method of improving mean skin clarity in a human, the method comprising topically administering the product composition according to claim 1 to the skin of the human.

30. The method of claim 29, wherein the product composition is administered once a day for a period of 2, 4, or 8 weeks.

31. The method of claim 29, wherein the mean skin clarity is improved by at least 1%.
